(19) United States
(12) Patent Application Publication
(10) Pub. No.: US 2023/0048492 A1
(43) Pub. Date: Feb. 16, 2023

(54) Adeno-Associated Viral (AAV) Vectors for Tissue-Targeted Expression of Therapeutic Genes

(71) Applicant: The Brigham and Women`s Hospital, Inc., Boston, MA (US)

(72) Inventor: Fengfeng Bei, West Roxbury, MA (US)

(21) Appl. No.: 17/845,225

(22) Filed: Jun. 21, 2022

Related U.S. Application Data
(60) Provisional application No. 63/213,045, filed on Jun. 21, 2021.

(51) Int. Cl. C12N 15/86; C12N 15/62; C07K 14/34; C07K 14/525; C07K 14/755; A61P 35/00

(52) U.S. Cl. CPC C12N 15/86; C12N 15/625; C07K 14/34; C07K 14/525; C07K 14/755; A61P 35/00

(57) ABSTRACT

Described herein are compositions and methods for tissue-targeted expression of therapeutic genes, using AAV expression vectors that reduce the risk of toxicity associated with AAV gene therapy in the CNS by de-targeting the vulnerable neurons cells including the DRG cells on gene expression, and de-targeting the liver, a major suspect for over-expression in the periphery.

### CROSS-REFERENCE TO RELATED APPLICATIONS

This application claims the benefit of U.S. Provisional Patent Application No. 63/213,045, filed Jun. 21, 2021. The entire contents of the foregoing are incorporated herein by reference.

### SEQUENCE LISTING

This application contains a Sequence Listing that has been submitted electronically as an ASCII text file named “40175-0036001_SL_ST25.txt.” The ASCII text file, created on Sep. 2, 2022, is 47,797 bytes in size. The material in the ASCII text file is hereby incorporated by reference in its entirety.

### TECHNICAL FIELD

Described herein are compositions and methods for tissue-targeted expression of therapeutic genes, using AAV expression vectors.

### BACKGROUND

For tissue-targeted gene therapy, e.g., in the CNS, it is desirable to use a vector that is targeted to the specific tissue and has reduced expression in non-target tissues.

### SUMMARY

Provided herein are AAV vectors comprising an AAV capsid, wherein the AAV capsid comprises a peptide insert of up to 21 amino acids, and wherein the peptide insert comprises a targeting peptide as described herein, e.g., 5-7 amino acids of a peptide shown in Table A, e.g., TVSALFK (SEQ ID NO: 36); a microRNA targeting sequence, preferably at the 3′UTR; and optionally a transgene, e.g., a therapeutic transgene. In some embodiments, the peptide insert comprises TVSALFK (SEQ ID NO:36)(also referred to herein as targeting peptide CPP.21). In some embodiments, the peptide insert comprises TVSALK (SEQ ID NO:32) (also referred to herein as targeting peptide CPP.16). In some embodiments, the peptide insert consists of TVSALFK (SEQ ID NO: 36). In some embodiments, the peptide insert consists of TVSALK (SEQ ID NO: 32). In some embodiments, the AAV vector comprises a transgene sequence. In some embodiments, the transgene sequence encodes a therapeutic agent (e.g., a therapeutic transgene).

In some embodiments, the AAV vector comprises a non-coding RNA, e.g., an shRNA, siRNA or miRNA.

In some embodiments, delivery of the transgene sequence or the non-coding RNA to an organ or tissue is enhanced relative to an AAV vector comprising an AAV capsid without the peptide insert and the transgene sequence or the non-coding RNA. In some embodiments, the organ or tissue comprises permeability barriers. In some embodiments, the organ or tissue comprises epithelium comprising tight junctions. In some embodiments, the organ or tissue is the brain or central nervous system.

Also provided are compositions comprising the AAV vector of claim 2, and a pharmaceutically acceptable carrier.

Further, provided herein are adeno-associated virus (AAV) vectors, preferably comprising a target tissue-tropic capsid (e.g., comprising a targeting peptide, e.g., comprising 5-7 amino acids of a peptide shown in Table A, e.g., TVSALFK (SEQ ID NO:36), optionally AAV.CPP.16, AAV.CPP.21 or AAV9, and further comprising an expression cassette comprising, preferably from 5′ to 3′: a promoter that drives expression in cells of the target tissue, an optional secretory signal peptide sequence, a coding sequence for a protein of interest, an optional Woodchuck Hepatitis Virus Posttranscriptional Regulatory Element (WPRE), a polyA signal sequence, and at least one microRNA targeting sequence selected from a microRNA 122 targeting sequence (miR-122T), e.g., targeting the 5p strand, e.g., comprising CAAACACCATTGTCACACTCCA (SEQ ID NO: 21); a microRNA 124 targeting sequence (miR-124T), e.g., targeting the 5p strand, e.g., comprising ATCAAGGTCCGCTGTGAACACG (SEQ ID NO: 20); a microRNA 200c targeting sequence (miR-200cT), e.g., targeting the 5p strand, e.g., CCAAACACTGCTGGGTAAGACG (SEQ ID NO: 22); and/or microRNA 1 targeting sequence (miR-1T), e.g., targeting the 5p strand, e.g., ATGGGCATATAAAGAAGTATGT (SEQ ID NO: 24), at the 3′ UTR. In some embodiments, the peptide insert comprises TVSALFK (SEQ ID NO:36). In some embodiments, the peptide insert comprises TVSALK (SEQ ID NO:32). In some embodiments, the peptide insert consists of TVSALFK (SEQ ID NO: 36). In some embodiments, the peptide insert consists of TVSALK (SEQ ID NO: 32).

In some embodiments, the capsid is CNS tropic, e.g., neuronal or glial-tropic.

In some embodiments, the promoter that drives expression in the CNS drives expression in neuronal cells or glial cells. In some embodiments, the promoter that drives expression in glial cells is a GFAP promoter, gfaABC₁D promoter, gfa2 promoter, ALDH1L1 promoter, SLC1A3 promoter, Gjb6 promoter, Mbp promoter, MAG promoter, CBh promoter, F4/80 promoter, CD68 promoter, or CD11B promoter.

In some embodiments, the promoter that drives expression in neuronal cells is a neuronal-specific enolase (NSE) promoter, Synapsin promoter, calcium/calmodulin-dependent protein kinase II promoter, tubulin alpha 1 promoter, platelet-derived growth factor beta chain promoter, parvalbumin promoter, GAD67 promoter or CCK promoter.

In some embodiments, the promoter is a ubiquitous promoter, optionally major immediate early human cytomegalovirus promoter (MIEhCMV), Chicken β-Actin Promoter (CBA); Human Cytomegalovirus Immediate/Early Gene Promoter and Enhancer (CMV); Chicken β-Actin/Cytomegalovirus Hybrid Promoter (CAG); Rous Sarcoma Virus Long Terminal Repeat Promoter (RSV); SV40 promoter; EF1alpha promoter.

In some embodiments, the AAV vectors further comprise an enhancer, optionally CMV-Enhancer, mD1x enhancer, AQP4 enhancer.

In some embodiments, the optional secretory signal peptide sequence is a Human IL-2 signal peptide (optionally MYRMQLLSCIALSLALVTNS, SEQ ID NO: 16), human albumin signal peptide, human alpha 1-antitrypsin signal peptide, or human factor VIII signal peptide.

In some embodiments, the polyA signal sequence is from human growth hormone (hGH), SV40, bovine growth hormone (bGH), or beta-globin, e.g., rabbit beta-globin (rbGlob).

In some embodiments, the AAV vectors include a plurality of, e.g., 2-10 or 3-5, microRNA targeting sequences, optionally separated by spacer sequence (e.g., of 1-50 nucleotides).

In some embodiments, the protein of interest is a therapeutic protein. In some embodiments, the therapeutic protein of interest is an antibody such as immune checkpoint inhibitors.

In some embodiments, the therapeutic protein of interest is a toxin, a suicide protein, or an antibody. In some embodiments, the toxin is diphtheria toxin, tumor necrosis factor-related apoptosis-inducing ligand (TRAIL), or TNF-α. In some embodiments, the suicide protein is herpes simplex virus thymidine kinase (HSVTK), bacterial or fungal cytosine deaminase (CD), carboxypeptidase G2 (CPG2), nitroreductase (NTR), Cytochrome P450 (CYP), purine nucleoside phosphorylase (PNP), horseradish peroxidase (HRP), or carboxylesterase (CE).

Also provided herein are methods of directing expression of a protein of interest in a cell in a target tissue, preferably without substantial expression of the protein of interest in non-target cells, the method comprising introducing an AAV vector as described herein into the cell.

Additionally provided are methods of inducing cell death in a cell, e.g., in a glial cell or a neuronal cell, the method comprising introducing an AAV vector as described herein into the cell, preferably wherein the therapeutic protein of interest is a toxin, a suicide protein, or an antibody. In some embodiments, the therapeutic protein of interest is a suicide protein, and the method further comprises contact the cell with a nontoxic prodrug that is a substrate for the suicide protein, wherein action of the suicide protein on the nontoxic prodrug results in production of a toxic metabolite that induces cell death. In some embodiments, the suicide protein is herpes simplex virus thymidine kinase (HSVTK) and the nontoxic prodrug is ganciclovir (GCV); the suicide protein is cytosine deaminase (CD) and the nontoxic prodrug is 5-flourouracil (5-FU); the suicide protein is carboxypeptidase G2 (CPG2) and the nontoxic prodrug is nitrogen mustard (NM) or a derivate thereof such as ZD2767P or CMDA (4-[(2-chloroethyl)(2-mesyloxyethyl)amino]benzoyl-L-glutamicacid); the suicide protein is nitroreductase (NTR) and the nontoxic prodrug is CB1954 or an analog thereof; the suicide protein is Cytochrome P450 (CYP) and the nontoxic prodrug is an oxazaphosphorine drug such as cyclophosphamide (CPA) and ifosfomide (IFO); the suicide protein is purine nucleoside phosphorylase (PNP) and the nontoxic prodrug is 6-Methylpurine Deoxyriboside or an analog thereof, e.g., fludarabine phosphate (F-araAMP) or 2-fluoro-2-deoxyadenosine (F-dAdo); the suicide gene is horseradish peroxidase (HRP) and the nontoxic prodrug is indole-3-acetic acid (HRP/IAA); or the suicide protein is carboxylesterase (CE) and the nontoxic prodrug is irinotecan.

In some embodiments, the glial cell is a cancer cell in a subject. In some embodiments, the cancer is glioblastoma.

In some embodiments, the AAV vector is a vector that targets the CNS; wherein the capsid is AAV.CPP.16 or AAV.CPP.21; the promoter is a GFAP promoter or a Syn promoter; and the microRNA targeting sequences target one, two or all three of microRNA 122, microRNA 200c and microRNA 1. In some embodiments, the protein of interest is MeCP2, CNTF, or NGF.

In some embodiments, the AAV vector is a vector that targets the Liver; wherein the capsid is AAV.CPP.16 or AAV9; the promoter is a LSP promoter or an al-antitrypsin promoter; the microRNA targeting sequences target one, two or all three of microRNA 124, microRNA 200c and microRNA 1. In some embodiments, the protein of interest is Factor VIII or factor IX.

In some embodiments, the AAV vector is a vector that targets the heart or other muscle; wherein the capsid is AAV.CPP.16; the promoter is a MLC2v promoter or MCK promoter; and the microRNA targeting sequences target one, two or all three of microRNA 122, microRNA 124 and microRNA 200c. In some embodiments, the protein of interest is mini dystrophin or factor IX.

In some embodiments, the AAV vector is a vector that targets the lung, wherein the capsid is AAV.CPP.16; the promoter is an SP-B promoter or SP-C promoter; and the microRNA targeting sequences target microRNA one, two or all three of 122, microRNA 124 and microRNA 1. In some embodiments, the protein of interest is Alpha-1 antitrypsin or an anti-SARS antibody.

Also provided herein are methods of delivering a therapeutic transgene or therapeutic non-coding RNA to a cell of the central nervous system in a subject. The methods include administering an AAV vector as described herein, wherein the AAV vector comprises an AAV capsid, wherein the AAV capsid comprises a peptide insert of up to 21 amino acids, and wherein the peptide insert comprises a targeting peptide as described herein, e.g., 5-7 amino acids of TVSALFK (SEQ ID NO:36); a microRNA targeting sequence, preferably at the 3′UTR; and the therapeutic transgene or therapeutic non-coding RNA. In some embodiments, the peptide insert comprises TVSALFK (SEQ ID NO:36). In some embodiments, the peptide insert comprises TVSALK (SEQ ID NO:32). In some embodiments, the peptide insert consists of TVSALFK (SEQ ID NO: 36). In some embodiments, the peptide insert consists of TVSALK (SEQ ID NO: 32). In some embodiments, the method comprises delivery to the cortex, cerebellum, hippocampus, substantia nigra, or amygdala. In some embodiments, the method comprises delivery to neurons, astrocytes, glial cells, or cadiomyocytes. In some embodiments, the subject has Parkinson's disease, Alzheimer's disease, Huntington's disease, amyotrophic lateral sclerosis, multiple sclerosis, epilepsy, stroke, brain cancer, spinocerebellar ataxia, Canavan's disease, or brain cancer.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a schematic illustration of a tissue-targeting AAV vector as described herein.

FIGS. 2A-B are schematic illustrations of exemplary liver-detargeting, CNS-tropic gene therapy vectors, e.g., for glioblastoma. FIG. 2A: vector with 5× miR-122T array.

FIG. 2B: vector with a 5× miR-122T array, 5× miR-200cT array, and 5× miR-1T array.

FIG. 3 is a schematic illustration of an exemplary 5× miR-122T array design. Figure discloses SEQ ID NOS 111, 111, and 114, respectively, in order of appearance.

FIGS. 4A-B show that both AAV.CPP.16-GFAP-RFP and AAV.CPP.16-GFAP-RFP-miR122T transduce GL261 GBM tumor cells in vitro. FIG. 4A: fluorescent staining. FIG. 4B: mean fluorescence intensity.

FIGS. 5A-B show that both AAV.CPP.16-GFAP-RFP and AAV.CPP.16-GFAP-RFP-miR122T target the mouse brain after intravenous administration of 1e12 vg per animal. FIG. 5A: representative brain sections showing RFO-labeled cells (white dots).

FIG. 5B: quantification on averaged number of RFP-labeled cells. n.s.: no significant difference.

FIGS. 6A-B show reduced liver transduction by AAV.CPP.16-GFAP-RFP-miR122T after intravenous administration of 1e12 vg per animal. FIG. 6A: liver section showing RFP-labeled cells with DAPI nuclear counter-staining. FIG. 6B: percentages of RFP-labeled cells in the liver. ****: P<0.0001

FIG. 7 shows lung and heart transduction by AAV.CPP.16-GFAP-RFP-miR122T after intravenous administration of 1e12 vg per animal.

FIG. 8 is a schematic illustration of an exemplary “suicide” gene therapy design. Expression of thymidine kinase 1 turns ganciclovir (GCV) into cytotoxic drugs.

FIG. 9 shows AAV-GFAP-TK1-miR-122T biodistribution in brain and liver using HA as tag (shown in green).

FIGS. 10A-B show “Suicide” gene therapy in glioblastoma. FIG. 10A: experimental timeline. FIG. 10B: representative serial sections showing the tumor size in animals treated with AAV/GCV or control (PBS).

FIGS. 11A-C show “Suicide” gene therapy in glioblastoma. FIG. 11A: experimental timeline. FIG. 11B: bar chart of tumor size in animals treated with AAV/GCV or control (PBS). FIG. 11C: Kaplan-Meier survival curve showing survival outcomes of animals treated with AAV/GCV or control (PBS).

### DETAILED DESCRIPTION

CNS-targeted gene therapies can use cells inside the CNS as a “bio-factory” to produce potentially therapeutic proteins such as growth factors and antibodies, or anti-cancer therapeutics. Since most cells in the CNS are not evolved to mass-produce foreign transgenes, one approach to expressing transgenes would be to target particular CNS cell types that are capable of expressing transgenes with high efficiency and good tolerance. Transcriptional regulatory elements such as promoters and microRNAs have been employed to achieve cell type specificity for expression of transgenes with varying levels of success. Described herein are combinations of regulatory elements that comprise optimal expression cassettes. These cassettes possess advantageous characteristics for CNS application including 1) high efficiency of transgene expression in the CNS; 2) few side effects on vulnerable cell populations such as the dorsal root ganglion (DRG) cells; 3) reduced exposure of peripheral tissues as a result of inhibiting expression in the periphery such as in the liver.

Tissue specificity of AAV-mediated gene expression can be achieved by applying a combination of vector capsids and expression regulatory elements including promoters and microRNAs.

Described herein are adeno-associated virus (AAV) vectors comprising an AAV capsid, wherein the AAV capsid comprises a peptide insert of up to 21 amino acids, and wherein the peptide insert comprises a targeting peptide as described herein, e.g., 5-7 amino acids of TVSALFK (SEQ ID NO:36), and a microRNA targeting sequence, preferably at the 3′UTR. Also provided herein are AAV-based expression cassettes designed for tissue targeted, e.g., CNS, gene therapy. The cassettes can contain a promoter, followed by a secretory signal peptide sequence, the coding sequence for a therapeutic protein of interest, an optional Woodchuck Hepatitis Virus Posttranscriptional Regulatory Element (WPRE), a polyA signal sequence, and finally a microRNA targeting sequence at the 3′ UTR. The microRNA targeting sequence reduces expression in non-target tissues. See, e.g., Xie et al., Mol Ther. 2011 March;19(3):526-35.

AAV and Targeting Peptides

A preferred viral vector system useful for delivery of nucleic acids in the present methods is the adeno-associated virus (AAV). AAV is a tiny non-enveloped virus having a 25 nm capsid. No disease is known or has been shown to be associated with the wild type virus. AAV has a single-stranded DNA (ssDNA) genome. AAV has been shown to exhibit long-term episomal transgene expression, and AAV has demonstrated excellent transgene expression in the brain, particularly in neurons. Vectors containing as little as 300 base pairs of AAV can be packaged and can integrate. Space for exogenous DNA is limited to about 4.7 kb. An AAV vector such as that described in Tratschin et al., Mol. Cell. Biol. 5:3251-3260 (1985) can be used to introduce DNA into cells. A variety of nucleic acids have been introduced into different cell types using AAV vectors (see for example Hermonat et al., Proc. Natl. Acad. Sci. USA 81:6466-6470 (1984); Tratschin et al., Mol. Cell. Biol. 4:2072-2081 (1985); Wondisford et al., Mol. Endocrinol. 2:32-39 (1988); Tratschin et al., J. Virol. 51:611-619 (1984); and Flotte et al., J. Biol. Chem. 268:3781-3790 (1993). There are numerous alternative AAV variants (over 100 have been cloned), and AAV variants have been identified based on desirable characteristics.

In some embodiments, the AAV include targeting peptides that enhance permeation through the BBB, e.g., when inserted into the capsid of an AAV, e.g., AAV1, AAV2, AAV8, or AAV9, or when conjugated to a biological agent, e.g., an antibody or other large biomolecule, either chemically or via expression as a fusion protein.

In some embodiments, the targeting peptides comprise sequences of at least 5 amino acids. In some embodiments, the amino acid sequence comprises at least 4, e.g., 5, contiguous amino acids of the sequences VPALR (SEQ ID NO:29) or VSALK (SEQ ID NO:30).

In some embodiments, the targeting peptides comprise a sequence of X₁ X₂ X₃ X₄ X₅, wherein:
(i) X₁, X₂, X₃, X₄ are any four non-identical amino acids of V, A, L, I, G, P, S, T, or M; and
(ii) X₅ is K, R, H, D, or E (SEQ ID NO:97).

In some embodiments, the targeting peptides comprise sequences of at least 6 amino acids. In some embodiments, the amino acid sequence comprises at least 4, e.g., 5 or 6 contiguous amino acids of the sequences TVPALR (SEQ ID NO:31), TVSALK (SEQ ID NO:32), TVPMLK (SEQ ID NO:4[[1]]0) and TVPTLK (SEQ ID NO:41).

In some embodiments, the targeting peptides comprise a sequence of X₁ X₂ X₃ X₄ X₅ X₆, wherein:
(i) X₁ is T;
(ii) X₂, X₃, X₄, X₅ are any four non-identical amino acids of V, A, L, I, G, P, S, T, or M; and
(iii) X₆ is K, R, H, D, or E (SEQ ID NO:98).

In some embodiments, the targeting peptides comprise a sequence of X₁ X₂ X₃ X₄ X₅ X₆, wherein:
(i) X₁, X₂, X₃, X₄ are any four non-identical amino acids from V, A, L, I, G, P, S, T, or M;
(ii) X₅ is K, R, H, D, or E; and
(iii) X₆ is E or D (SEQ ID NO:99).

In some embodiments, the targeting peptides comprise sequences of at least 7 amino acids. In some embodiments, the amino acid sequence comprises at least 4, e.g., 5, 6, or 7 contiguous amino acids of the sequences FTVSALK (SEQ ID NO:33), LTVSALK (SEQ ID NO:34), TVSALFK (SEQ ID NO:36), TVPALFR (SEQ ID NO:37), TVPMLFK (SEQ ID NO:38) and TVPTLFK (SEQ ID NO:39). In some other embodiments, the targeting peptides comprise a sequence of X₁ X₂ X₃ X₄ X₅ X₆ X₇, wherein:
(i) X₁ is F, L, W, or Y;
(ii) X₂ is T;
(iii) X₃, X₄, X₅, X₆ are any four non-identical amino acids of V, A, L, I, G, P, S, T, or M; and
(iv) X₇ is K, R, H, D, or E (SEQ ID NO:100).

In some embodiments, the targeting peptides comprise a sequence of X₁ X₂ X₃ X₄ X₅ X₆ X₇, wherein:
(i) X₁ is T;
(ii) X₂, X₃, X₄, X₅ are any four non-identical amino acids of V, A, L, I, G, P, S, T, or M;
(iii) X₆ is K, R, H, D, or E; and
(iv) X₇ is E or D (SEQ ID NO:101).

In some embodiments, the targeting peptides comprise a sequence of X₁ X₂ X₃ X₄ X₅ X₆ X₇, wherein:
(i) X₁, X₂, X₃, X₄ are any four non-identical amino acids of V, A, L, I, G, P, S, T, or M;
(ii) X₅ is K, R, H, D, or E;
(iii) X₆ is E or D; and
(iv) X₇ is A or I (SEQ ID NO:102).

In some embodiments, the targeting peptides comprise a sequence of V[S/p][A/m/t/]L (SEQ ID NO:103), wherein the upper case letters are preferred at that position. In some embodiments, the targeting peptides comprise a sequence of TV[S/p][A/m/t/]L (SEQ ID NO:104). In some embodiments, the targeting peptides comprise a sequence of TV[S/p][A/m/t/]LK (SEQ ID NO:105). In some embodiments, the targeting peptides comprise a sequence of TV[S/p][A/m/t/]LFK. (SEQ ID NO:106).

In some embodiments, the targeting peptide does not consist of VPALR (SEQ ID NO:29) or VSALK (SEQ ID NO:30).

In some embodiments, the peptide insert comprises TVSALFK (SEQ ID NO:36). In some embodiments, the peptide insert comprises TVSALK (SEQ ID NO:32). In some embodiments, the peptide insert consists of TVSALFK (SEQ ID NO: 36). In some embodiments, the peptide insert consists of TVSALK (SEQ ID NO: 32).

Specific exemplary amino acid sequences that include the above mentioned 5, 6, or 7-amino acid sequences are listed in Table A.

TABLE A
Targeting Sequences

| SEQ ID NO: | Targeting Peptide Sequence |
|---|---|
| 29. | VPALR |
| 30. | VSALK |
| 31. | TVPALR |
| 32. | TVSALK |
| 33. | FTVSALK |
| 34. | LTVSALK |
| 35. | TFVSALK |
| 36. | TVSALFK |
| 37. | TVPALFR |
| 38. | TVPMLFK |
| 39. | TVPTLFK |
| 40. | TVPMLK |
| 41. | TVPTLK |
| 42. | VPMLK |
| 43. | VPTLK |
| 44. | VPMLKE |
| 45. | VPTLKD |
| 46. | VPALRD |
| 47. | VSALKE |
| 48. | VSALKD |
| 49. | TAVSLK |
| 50. | TALVSK |
| 51. | TVLSAK |
| 52. | TLVSAK |
| 53. | TMVPLK |
| 54. | TMLVPK |
| 55. | TVLPMK |
| 56. | TLVPMK |
| 57. | TTVPLK |
| 58. | TTLVPK |
| 59. | TVLPTK |
| 60. | TLVPTK |
| 61. | TAVPLR |
| 62. | TALVPR |
| 63. | TVLPAR |
| 64. | TLVPAR |
| 65. | TAVSLKE |
| 66. | TALVSKE |
| 67. | TVLSAKE |
| 68. | TLVSAKE |
| 69. | TMVPLKE |
| 70. | TMLVPKE |
| 71. | TVLPMKE |
| 72. | TLVPMKE |
| 73. | TTVPLKD |
| 74. | TTLVPKD |
| 75. | TVLPTKD |
| 76. | TLVPTKD |
| 77. | TAVPLRD |
| 78. | TALVPRD |
| 79. | TVLPARD |
| 80. | TLVPARD |
| 81. | TAVSLFK |
| 82. | TALVSFK |
| 83. | TVLSAFK |
| 84. | TLVSAFK |
| 85. | TMVPLFK |
| 86. | TMLVPFK |
| 87. | TVLPMFK |
| 88. | TLVPMFK |
| 89. | TTVPLFK |
| 90. | TTLVPFK |
| 91. | TVLPTFK |
| 92. | TLVPTFK |
| 93. | TAVPLFR |
| 94. | TALVPFR |
| 95. | TVLPAFR |
| 96. | TLVPAFR |

Targeting peptides including reversed sequences can also be used, e.g., KLASVT (SEQ ID NO:107) and KFLASVT (SEQ ID NO:108). Preferred AAV for use in the present methods and compositions include AAV.CPP.16 and AAV.CPP.21 (described in WO 2020/014471). Other AAV as known in the art (e.g., AAV1, 2, 3, 4, 5, 6, 7, 8 and variants thereof and others as known in the art or described herein) can also be used. AAV.CPP.16 can be used for targeting CNS, liver, heart/muscle, and/or lung.

Exemplary VP1 protein sequences for AAV9, AAV.CPP.16 and AAV.CPP.21 are provided as below:

| AAV9 | MAADGYLPDWLEDNLSEGIREWWALKPGAPQPKANQQHQDNARGLVLPGYKYLGPGNGLD 60 |
|---|---|
| AAV.CPP16 | MAADGYLPDWLEDNLSEGIREWWALKPGAPQPKANQQHQDNARGLVLPGYKYLGPGNGLD 60 |
| AAV.CPP21 | MAADGYLPDWLEDNLSEGIREWWALKPGAPQPKANQQHQDNARGLVLPGYKYLGPGNGLD 60 |
| AAV9 | KGEPVNAADAAALEHDKAYDQQLKAGDNPYLKYNHADAEFQERLKEDTSFGGNLGRAVFQ 120 |
| AAV.CPP16 | KGEPVNAADAAALEHDKAYDQQLKAGDNPYLKYNHADAEFQERLKEDTSFGGNLGRAVFQ 120 |
| AAV.CPP21 | KGEPVNAADAAALEHDKAYDQQLKAGDNPYLKYNHADAEFQERLKEDTSFGGNLGRAVFQ 120 |
| AAV9 | AKKRLLEPLGLVEEAAKTAPGKKRPVEQSPQEPDSSAGIGKSGAQPAKKRLNFGQTGDTE 180 |
| AAV.CPP16 | AKKRLLEPLGLVEEAAKTAPGKKRPVEQSPQEPDSSAGIGKSGAQPAKKRLNFGQTGDTE 180 |
| AAV.CPP21 | AKKRLLEPLGLVEEAAKTAPGKKRPVEQSPQEPDSSAGIGKSGAQPAKKRLNFGQTGDTE 180 |
| AAV9 | SVPDPQPIGEPPAAPSGVGSLTMASGGGAPVADNNEGADGVGSSSGNWHCDSQWLGDRVI 240 |
| AAV.CPP16 | SVPDPQPIGEPPAAPSGVGSLTMASGGGAPVADNNEGADGVGSSSGNWHCDSQWLGDRVI 240 |
| AAV.CPP21 | SVPDPQPIGEPPAAPSGVGSLTMASGGGAPVADNNEGADGVGSSSGNWHCDSQWLGDRVI 240 |
| AAV9 | TTSTRTWALPTYNNHLYKQISNSTSGGSSNDNAYFGYSTPWGYFDFNRFHCHFSPRDWQR 300 |
| AAV.CPP16 | TTSTRTWALPTYNNHLYKQISNSTSGGSSNDNAYFGYSTPWGYFDFNRFHCHFSPRDWQR 300 |
| AAV.CPP21 | TTSTRTWALPTYNNHLYKQISNSTSGGSSNDNAYFGYSTPWGYFDFNRFHCHFSPRDWQR 300 |
| AAV9 | LINNNWGFRPKRLNFKLFNIQVKEVTDNNGVKTIANNLTSTVQVFTDSDYQLPYVLGSAH 360 |
| AAV.CPP16 | LINNNWGFRPKRLNFKLFNIQVKEVTDNNGVKTIANNLTSTVQVFTDSDYQLPYVLGSAH 360 |
| AAV.CPP21 | LINNNWGFRPKRLNFKLFNIQVKEVTDNNGVKTIANNLTSTVQVFTDSDYQLPYVLGSAH 360 |
| AAV9 | EGCLPPFPADVFMIPQYGYLTLNDGSQAVGRSSFYCLEYFPSQMLRTGNNFQFSYEFENV 420 |
| AAV.CPP16 | EGCLPPFPADVFMIPQYGYLTLNDGSQAVGRSSFYCLEYFPSQMLRTGNNFQFSYEFENV 420 |
| AAV.CPP21 | EGCLPPFPADVFMIPQYGYLTLNDGSQAVGRSSFYCLEYFPSQMLRTGNNFQFSYEFENV 420 |
| AAV9 | PFHSSYAHSQSLDRLMNPLIDQYLYYLSKTINGSGQNQQTLKFSVAGPSNMAVQGRNYIP 480 |
| AAV.CPP16 | PFHSSYAHSQSLDRLMNPLIDQYLYYLSKTINGSGQNQQTLKFSVAGPSNMAVQGRNYIP 480 |
| AAV.CPP21 | PFHSSYAHSQSLDRLMNPLIDQYLYYLSKTINGSGQNQQTLKFSVAGPSNMAVQGRNYIP 480 |
| AAV9 | GPSYRQQRVSTTVTQNNNSEFAWPGASSWALNGRNSLMNPGPAMASHKEGEDRFFPLSGS 540 |
| AAV.CPP16 | GPSYRQQRVSTTVTQNNNSEFAWPGASSWALNGRNSLMNPGPAMASHKEGEDRFFPLSGS 540 |
| AAV.CPP21 | GPSYRQQRVSTTVTQNNNSEFAWPGASSWALNGRNSLMNPGPAMASHKEGEDRFFPLSGS 540 |
| AAV9 | LIFGKQGTGRDNVDADKVMITNEEEIKTTNPVATESYGQVATNHQSAQ-------AQAQT 593 |
| AAV.CPP16 | LIFGKQGTGRDNVDADKVMITNEEEIKTTNPVATESYGQVATNHQSAQTVSAL-KAQAQT 599 |
| AAV.CPP21 | LIFGKQGTGRDNVDADKVMITNEEEIKTTNPVATESYGQVATNHQSAQTVSALFKAQAQT 600 |
| AAV9 | GWVQNQGILPGMVWQDRDVYLQGPIWAKIPHTDGNFHPSPLMGGFGMKHPPPQILIKNTP 653 |
| AAV.CPP16 | GWVQNQGILPGMVWQDRDVYLQGPIWAKIPHTDGNFHPSPLMGGFGMKHPPPQILIKNTP 659 |
| AAV.CPP21 | GWVQNQGILPGMVWQDRDVYLQGPIWAKIPHTDGNFHPSPLMGGFGMKHPPPQILIKNTP 660 |
| AAV9 | VPADPPTAFNKDKLNSFITQYSTGQVSVEIEWELQKENSKRWNPEIQYTSNYYKSNNVEF 713 |
| AAV.CPP16 | VPADPPTAFNKDKLNSFITQYSTGQVSVEIEWELQKENSKRWNPEIQYTSNYYKSNNVEF 719 |
| AAV.CPP21 | VPADPPTAFNKDKLNSFITQYSTGQVSVEIEWELQKENSKRWNPEIQYTSNYYKSNNVEF 720 |
| (SEQ ID NO: 1) | |
| AAV9 | AVNTEGVYSEPRPIGTRYLTRNL 736 |
| (SEQ ID NO: 2) | |
| AAV.CPP16 | AVNTEGVYSEPRPIGTRYLTRNL 742 |
| (SEQ ID NO: 3) | |
| AAV.CPP21 | AVNTEGVYSEPRPIGTRYLTRNL 743 |

In some embodiments, the AAV also has one or more additional mutations that increase delivery to the target tissue, e.g., the CNS, or that reduce off-tissue targeting, e.g., mutations that decrease liver delivery when CNS, heart, or muscle delivery is intended (e.g., as described in Pulicherla et al. (2011) Mol Ther 19:1070-1078); or the addition of other targeting peptides, e.g., as described in Chen et al. (2008) Nat Med 15:1215-1218 or Xu et al., (2005) Virology 341:203-214 or U.S. Pat. Nos. 9,102,949; 9,585,971; and US20170166926. See also Gray and Samulski (2011) “Vector design and considerations for CNS applications,” in Gene Vector Design and Application to Treat Nervous System Disorders ed. Glorioso J., editor. (Washington, D.C.: Society for Neuroscience) 1-9, available at sfn.org/˜/media/SfN/Documents/Short %20Courses/2011%20 Short %20Course %20I/2011_SC1_Gray.ashx.

Regulatory Sequences/Promoters

The virus can also include one or more sequences that promote expression of a transgene, e.g., one or more promoter sequences; enhancer sequences, e.g., 5′ untranslated region (UTR) or a 3′ UTR; a polyadenylation site; and/or insulator sequences. In some embodiments, the promoter is a brain tissue specific promoter, e.g., a neuron-specific or glia-specific promoter. In certain embodiments, the promoter is a promoter of a gene selected to from: neuronal nuclei (NeuN), glial fibrillary acidic protein (GFAP), MeCP2, adenomatous polyposis coli (APC), ionized calcium-binding adapter molecule 1 (Iba-1), synapsin I (SYN), calcium/calmodulin-dependent protein kinase II, tubulin alpha I, neuron-specific enolase and platelet-derived growth factor beta chain. In some embodiments, the promoter is a pan-cell type promoter, e.g., cytomegalovirus (CMV), beta glucuronidase, (GUSB), ubiquitin C (UBC), or rous sarcoma virus (RSV) promoter. GRP78 or HMGB2 promoters can also be used.

In some embodiments, the promoter drives expression in neuronal cells or glial cells. For example, a promoter that drives expression in glial cells can be a GFAP promoter, gfaABC1D promoter, gfa2 promoter, ALDH1L1 promoter, SLC1A3 promoter, Gjb6 promoter, Mbp promoter, MAG promoter, CBh promoter, F4/80 promoter, CD68 promoter, or CD11B promoter. As another example, a promoter that drives expression in neuronal cells can be a neuronal-specific enolase (NSE) promoter, Synapsin promoter, calcium/calmodulin-dependent protein kinase II promoter, tubulin alpha 1 promoter, platelet-derived growth factor beta chain promoter, parvalbumin promoter, GAD67 promoter, or CCK promoter.

In some embodiments, the promoter is a ubiquitous promoter, optionally major immediate early human cytomegalovirus promoter (MIEhCMV), Chicken β-Actin Promoter (CBA); Human Cytomegalovirus Immediate/Early Gene Promoter and Enhancer (CMV); Chicken β-Actin/Cytomegalovirus Hybrid Promoter (CAG); Rous Sarcoma Virus Long Terminal Repeat Promoter (RSV); SV40 promoter; EF1alpha promoter.

Synthetic promoters are also known, see, e.g., Jüttner et al., Nature Neuroscience volume 22, pages 1345-1356(2019); Morelli et al., J Gen Virol. 1999 March;80 (Pt 3):571-583; O'Carroll et al., Front Mol Neurosci. 2020; 13: 618020 (review).

The AAV vector can also include an enhancer, such as a CMV Enhancer, mD1x enhancer, or AQP4 enhancer. See, e.g., WO2020168279, Nair et al., iScience. 2020 Mar. 27; 23(3): 100888, Gruh et al., J Gene Med. 2008 January; 10(1): 21-32, Abe et al., FEBS Lett. 2017 December; 591(23): 3906-3915, and Dimidschstein et al., Nat Neurosci. 2016 December; 19(12): 1743-1949.

The AAV vector can also include a Woodchuck Hepatitis Virus Posttranscriptional Regulatory Element (WPRE) or alternatives, e.g., as described in PCT/EP2014/072852.

Preferably, the vector includes a polyA signal sequence at the 3′ end of the coding sequence for the transgene. Exemplary polyA signal sequence include human growth hormone (hGH), SV40, bovine growth hormone (bGH), or beta-globin, e.g., rabbit beta-globin (rbGlob).

Human growth hormone (hGH)
polyA signal-479 bp
(SEQ ID NO: 4)
ACGGGTGGCATCCCTGTGACCCCTCCCCAGTGCCT
CTCCTGGCCCTGGAAGTTGCCACTCCAGTGCCCAC
CAGCCTTGTCCTAATAAAATTAAGTTGCATCATTT
TGTCTGACTAGGTGTCCTTCTATAATATTATGGGG
TGGAGGGGGGTGGTATGGAGCAAGGGGCAAGTTGG
GAAGACAACCTGTAGGGCCTGCGGGGTCTATTGGG
AACCAAGCTGGAGTGCAGTGGCACAATCTTGGCTC
ACTGCAATCTCCGCCTCCTGGGTTCAAGCGATTCT
CCTGCCTCAGCCTCCCGAGTTGTTGGGATTCCAGG
CATGCATGACCAGGCTCAGCTAATTTTTGTTTTTT
TGGTAGAGACGGGGTTTCACCATATTGGCCAGGCT
GGTCTCCAACTCCTAATCTCAGGTGATCTACCCAC
CTTGGCCTCCCAAATTGCTGGGATTACAGGCGTGA
ACCACTGCTCCCTTCCCTGTCCTT
SV40 polyA signal-135 bp
(SEQ ID NO: 5)
AACTTGTTTATTGCAGCTTATAATGGTTACAAATA
AAGCAATAGCATCACAAATTTCACAAATAAAGCAT
TTTTTTCACTGCATTCTAGTTGTGGTTTGTCCAAA
CTCATCAATGTATCTTATCATGTCTGGATC
Bovine growth hormone (bGH)
polyA signal-228 bp
(SEQ ID NO: 6)
CGACTGTGCCTTCTAGTTGCCAGCCATCTGTTGTT
TGCCCCTCCCCCGTGCCTTCCTTGACCCTGGAAGG
TGCCACTCCCACTGTCCTTTCCTAATAAAATGAGG
AAATTGCATCGCATTGTCTGAGTAGGTGTCATTCT
ATTCTGGGGGGTGGGGTGGGGCAGGACAGCAAGGG
GGAGGATTGGGAAGACAATAGCAGGCATGCTGGGG
ATGCGGTGGGCTCTATGG
Rabbit beta-globin (rbGlob)
polyA signal-527 bp
(SEQ ID NO: 7)
TTCACTCCTCAGGTGCAGGCTGCCTATCAGAAGGT
GGTGGCTGGTGTGGCCAATGCCCTGGCTCACAAAT
ACCACTGAGATCTTTTTCCCTCTGCCAAAAATTAT
GGGGACATCATGAAGCCCCTTGAGCATCTGACTTC
TGGCTAATAAAGGAAATTTATTTTCATTGCAATAG
TGTGTTGGAATTTTTTGTGTCTCTCACTCGGAAGG
ACATATGGGAGGGCAAATCATTTAAAACATCAGAA
TGAGTATTTGGTTTAGAGTTTGGCAACATATGCCC
ATATGCTGGCTGCCATGAACAAAGGTTGGCTATAA
AGAGGTCATCAGTATATGAAACAGCCCCCTGCTGT
CCATTCCTTATTCCATAGAAAAGCCTTGACTTGAG
GTTAGATTTTTTTTATATTTTGTTTTGTGTTATTT
TTTTCTTTAACATCCCTAAAATTTTCCTTACATGT
TTTACTAGCCAGATTTTTCCTCCTCTCCTGACTAC
TCCCAGTCATAGCTGTCCCTCTTCTCTTATGGAGA
TC

Secretory Signal Sequence

A number of secretory signal peptide sequences are known in the art, including human signal sequences, examples of which are shown in Table 1 (Table adapted from novoprolabs.com/support/articles/commonly-used-leader-peptide-sequences-for-efficient-secretion-of-a-recombinant-protein-expressed-in-mammalian-cells-201804211337.html).

TABLE 1
Exemplary Human Secretory Signal
Peptide Sequences

| Human | | SEQ |
|---|---|---|
| Signal | | ID |
| sequence | Sequence | NO: |
| Oncostatin M | MGVLLTQRTLLSLV | 8 |
| | LALLFPSMASM | |
| IgG2 H | MGWSCIILFLVATATGVHS | 9 |
| Secrecon* | MWWRLWWLLLLLLLLWP | 10 |
| | MVWA | |
| IgKVIII | MDMRVPAQLLGLLLLW | 11 |
| | LRGARC | |
| CD33 | MPLLLLLPLLWAGALA | 12 |
| tPA | MDAMKRGLCCVLLLCGA | 13 |
| | VFVSPS | |
| Chymotrypsinogen | MAFLWLLSCWALLGTTFG | 14 |
| trypsinogen-2 | MNLLLILTFVAAAVA | 15 |
| Interleukin 2 | MYRMQLLSCIALSLALV | 16 |
| (IL-2) | TNS | |
| Albumin | MKWVTFISLLFSSAYS | 17 |
| (HSA) | | |
| insulin | MALWMRLLPLLALLAL | 18 |
| | WGPDPAAA | |
| alpha 1- | MPSSVSWGILLLAGLC | 19 |
| antitrypsin | CLVPVSLA | |

*Barash et al., Biochem Biophys Res Commun. Jun. 21, 2002; 294(4):835-42.

In some embodiments, another secretory sequence that promotes secretion is used, e.g., as described in von Heijne, J Mol Biol. 1985 Jul. 5;184(1):99-105; Kober et al., Biotechnol. Bioeng. 2013; 110: 1164-1173; Tsuchiya et al., Nucleic Acids Research Supplenzent No. 3 261-262 (2003).

microRNA Targeting Sequences

The AAVs described herein include one or more, e.g., a plurality, e.g., 2-10, 2-8, 2-5, or 3-5, microRNA targeting sequences, optionally separated by spacer sequence (e.g., of 1-50 nucleotides, e.g., comprising a spacer sequence that is not expected to pair with DNA of the targeting sequences to avoid nonspecific binding of miRNA). The following Table 2 provides exemplary miRNAs and their corresponding targeting sequences.

TABLE 2
Exemplary miRNA Sequences and Targeting Sequences

| | | | | miRNA | Targeting |
|---|---|---|---|---|---|
| mi- | | Tissue | Accession | Sequence | Sequence |
| RNA | Species | detargeted | (miRBase) | (5′-3′) | (5′-3′) |
| miR- | Mouse | Brain | MIMAT00 | CGUGUUCACA | ATCAAGGTCCG |
| 124a | | | 04527 | GCGGACCUUG | CTGTGAACACG |
| -5p | | | | AU | (SEQ ID |
| | | | | (SEQ ID NO: | NO: 110) |
| | | | | 113) | |
| miR- | Human | Brain | MIMAT00 | CGUGUUCACA | ATCAAGGTCCG |
| 124a | | | 04591 | GCGGACCUUG | CTGTGAACACG |
| -5p | | | | AU | (SEQ ID |
| | | | | (SEQ ID NO: | NO: 20) |
| | | | | 25) | |
| miR- | Mouse | Liver | MIMAT00 | UGGAGUGUGA | CAAACACCATT |
| 122- | | | 00246 | CAAUGGUGUU | GTCACACTCCA |
| 5p | | | | UG | (SEQ ID |
| | | | | (SEQ ID NO: | NO: 111) |
| | | | | 114) | |
| miR- | Human | Liver | MIMAT00 | UGGAGUGUGA | CAAACACCATT |
| 122- | | | 00421 | CAAUGGUGUU | GTCACACTCCA |
| 5p | | | | UG | (SEQ ID |
| | | | | (SEQ ID NO: | NO: 21) |
| | | | | 26) | |
| miR- | Mouse | Lung | MIMAT00 | CGUCUUACCC | CCAAACACTGC |
| 200c | | | 04663 | AGCAGUGUUU | TGGGTAAGACG |
| -5p | | | | GG | (SEQ ID |
| | | | | (SEQ ID NO: | NO: 112) |
| | | | | 115) | |
| miR- | Human | Lung | MIMAT00 | CGUCUUACCC | CCAAACACTGC |
| 200c | | | 04657 | AGCAGUGUUU | TGGGTAAGACG |
| -5p | | | | GG | (SEQ ID |
| | | | | (SEQ ID NO: | NO: 22) |
| | | | | 27) | |
| miR- | Mouse | Heart and | MIMAT00 | ACAUACUUCU | TATGGGCATAT |
| 1-5p | | Muscle | 16979 | UUAUAUGCCC | AAAGAAGTATGT |
| | | | | AUA | (SEQ ID |
| | | | | (SEQ ID | NO: 23) |
| | | | | NO: 28) | |
| miR- | Human | Heart and | MIMAT00 | ACAUACUUCU | ATGGGCATATA |
| 1-5p | | Muscle | 31892 | UUAUAUGCCC | AAGAAGTATGT |
| | | | | AU | (SEQ ID |
| | | | | (SEQ ID NO: | NO: 24) |
| | | | | 109) | |

In some embodiments, the AAV includes one, two, or three of a microRNA 122 targeting sequence (miR-122T), e.g., targeting the 5p strand, e.g., comprising CAAACACCATTGTCACACTCCA (SEQ ID NO: 21); a microRNA 124 targeting sequence (miR-124T), e.g., targeting the 5p strand, e.g., comprising ATCAAGGTCCGCTGTGAACACG (SEQ ID NO: 20); a microRNA 200c targeting sequence (miR-200cT), e.g., targeting the 5p strand, e.g., CCAAACACTGCTGGGTAAGACG (SEQ ID NO: 22); and/or microRNA 1 targeting sequence (miR-1T), e.g., targeting the 5p strand, e.g., ATGGGCATATAAAGAAGTATGT (SEQ ID NO: 24), at the 3′ UTR.

See also Qiao et al., Gene Ther. 2011 April; 18(4):403-10.

### Exemplary Embodiments

Tissue specificity of AAV-mediated gene expression can be achieved by applying a combination of vector capsids and expression regulatory elements including promoters and microRNAs. For example, for CNS selectivity, a set of miRNAs are used that de-target the liver, lung and/or heart. For muscle selectivity (such as for treating muscle diseases, e.g., Duchenne muscular dystrophy), miRNAs that de-target the liver, lung and the CNS are used. For conditions affecting the CNS and heart, e.g., for applications such as disease Friedreich ataxia, miRNAs that de-target the liver and the lung are used. Exemplary combinations are shown in Table 3. Additional exemplary transgenes are provided in Table B.

TABLE 3
Examples of tissue-tropic capsids, tissue-specific
promoters and microRNAs for use with CPP.16

| | Tissue-tropic | Tissue-specific | Tissue specific | Exemplary |
|---|---|---|---|---|
| Tissue | Capsids | promoter | microRNA | transgene |
| CNS | AAV.CPP.16, | GFAP promoter, | microRNA 9, | MeCP2, CNTF, |
| | AAV.CPP.21 | Syn promoter, etc. | microRNA 124a | NGF, etc. |
| Liver | AAV9, | LSP promoter, | microRNA 122 | Factor VIII, |
| | AAV.CPP.16 | α₁-antitrypsin | | factor IX, etc. |
| | | promoter, etc. | | |
| Heart | AAV.CPP.16 | MLC2v promoter, | microRNA 1, | Mini dystrophin, |
| (and | | MCK promoter, | microRNA 208 | factor IX, etc. |
| muscle) | | etc. | | |
| Lung | AAV.CPP.16 | SP-B promoter, | microRNA 200c | Alpha-1 |
| | | SP-C promoters, | | antitrypsin, |
| | | etc. | | SARS/COVID |
| | | | | antibodies, etc. |

CNTF, Ciliary Neurotrophic Factor;
NGF, Nerve growth factor;
MeCP2, Methyl-CpG Binding Protein 2

In some embodiments, the disclosure provides an AAV vector comprising an AAV capsid comprising a targeting peptide as described herein, e.g., 5-7 amino acids of TVSALFK (SEQ ID NO:36), and an expression cassette comprising a coding sequence for a protein of interest, a therapeutic transgene or non-coding RNA (e.g., as shown in Table B or Table 3), and at least one miR-9 targeting sequence as described herein. In some embodiments, the peptide insert comprises TVSALFK (SEQ ID NO:36). In some embodiments, the peptide insert comprises TVSALK (SEQ ID NO:32). In some embodiments, the peptide insert consists of TVSALFK (SEQ ID NO: 36). In some embodiments, the peptide insert consists of TVSALK (SEQ ID NO: 32). In some embodiments, the targeting peptides comprise a sequence of V[S/p][A/m/t/]L (SEQ ID NO:103), wherein the upper case letters are preferred at that position. In some embodiments, the targeting peptides comprise a sequence of TV[S/p][A/m/t/]L (SEQ ID NO:104). In some embodiments, the targeting peptides comprise a sequence of TV[S/p][A/m/t/]LK (SEQ ID NO:105). In some embodiments, the targeting peptides comprise a sequence of TV[S/p][A/m/t/]LFK. (SEQ ID NO:106). In some embodiments, the expression cassette comprises a CNS-specific promoter, e.g., a GFAP promoter or Syn promoter. In some embodiments, the coding sequence encodes GDNF, BDNF, AADC, Tau antibody, APP antibody, miRNA targeting HTT, shRNA targeting SOD, IFN-beta, Neuropeptide Y, IGF-1, osteopontin, HSV.TK1, PD-1/PD-L1 antibody, RNAi targeting ataxin, ASPA, ARSA, PSAP, MeCP2, CNTF, ATP7B, or NGF.

In some embodiments, the disclosure provides an AAV vector comprising an AAV capsid comprising a targeting peptide as described herein, e.g., 5-7 amino acids of TVSALFK (SEQ ID NO:36), and an expression cassette comprising a coding sequence for protein of interest, a therapeutic transgene or non-coding RNA (e.g., as shown in Table B or Table 3), and at least one miR-124a targeting sequence as described herein. In some embodiments, the peptide insert comprises TVSALFK (SEQ ID NO:36). In some embodiments, the peptide insert comprises TVSALK (SEQ ID NO:32). In some embodiments, the peptide insert consists of TVSALFK (SEQ ID NO: 36). In some embodiments, the peptide insert consists of TVSALK (SEQ ID NO: 32). In some embodiments, the targeting peptides comprise a sequence of V[S/p][A/m/t/]L (SEQ ID NO:103), wherein the upper case letters are preferred at that position. In some embodiments, the targeting peptides comprise a sequence of TV[S/p][A/m/t/]L (SEQ ID NO:104). In some embodiments, the targeting peptides comprise a sequence of TV[S/p][A/m/t/]LK (SEQ ID NO:105). In some embodiments, the targeting peptides comprise a sequence of TV[S/p][A/m/t/]LFK. (SEQ ID NO:106). In some embodiments, the expression cassette comprises a CNS-specific promoter, e.g., a GFAP promoter or Syn promoter. In some embodiments, the coding sequence encodes GDNF, BDNF, AADC, Tau antibody, APP antibody, miRNA targeting HTT, shRNA targeting SOD, IFN-beta, Neuropeptide Y, IGF-1, osteopontin, HSV.TK1, PD-1/PD-L1 antibody, RNAi targeting ataxin, ASPA, ARSA, PSAP, MeCP2, CNTF, ATP7B, or NGF.

In some embodiments, the disclosure provides an AAV vector comprising an AAV capsid comprising a targeting peptide as described herein, e.g., 5-7 amino acids of TVSALFK (SEQ ID NO:36), and an expression cassette comprising a coding sequence for a protein of interest, a therapeutic transgene or non-coding RNA (e.g., as shown in Table B or Table 3), and at least one miR-122 targeting sequence as described herein. In some embodiments, the peptide insert comprises TVSALFK (SEQ ID NO:36). In some embodiments, the peptide insert comprises TVSALK (SEQ ID NO:32). In some embodiments, the peptide insert consists of TVSALFK (SEQ ID NO: 36). In some embodiments, the peptide insert consists of TVSALK (SEQ ID NO: 32). In some embodiments, the targeting peptides comprise a sequence of V[S/p][A/m/t/]L (SEQ ID NO:103), wherein the upper case letters are preferred at that position. In some embodiments, the targeting peptides comprise a sequence of TV[S/p][A/m/t/]L (SEQ ID NO:104). In some embodiments, the targeting peptides comprise a sequence of TV[S/p][A/m/t/]LK (SEQ ID NO:105). In some embodiments, the targeting peptides comprise a sequence of TV[S/p][A/m/t/]LFK. (SEQ ID NO:106). In some embodiments, the expression cassette comprises a liver-specific promoter, e.g., a LSP promoter or al-antitrypsin promoter. In some embodiments, the coding sequence encodes ATP7B, UGT1A1, Factor VIII or factor IX.

In some embodiments, the disclosure provides an AAV vector comprising an AAV capsid comprising a targeting peptide as described herein, e.g., 5-7 amino acids of TVSALFK (SEQ ID NO:36), and an expression cassette comprising a coding sequence for protein of interest, a therapeutic transgene or non-coding RNA (e.g., as shown in Table B or Table 3), and at least one miR-1 targeting sequence as described herein. In some embodiments, the peptide insert comprises TVSALFK (SEQ ID NO:36). In some embodiments, the peptide insert comprises TVSALK (SEQ ID NO:32). In some embodiments, the peptide insert consists of TVSALFK (SEQ ID NO: 36). In some embodiments, the peptide insert consists of TVSALK (SEQ ID NO: 32). In some embodiments, the targeting peptides comprise a sequence of V[S/p][A/m/t/]L (SEQ ID NO:103), wherein the upper case letters are preferred at that position. In some embodiments, the targeting peptides comprise a sequence of TV[S/p][A/m/t/]L (SEQ ID NO:104). In some embodiments, the targeting peptides comprise a sequence of TV[S/p][A/m/t/]LK (SEQ ID NO:105). In some embodiments, the targeting peptides comprise a sequence of TV[S/p][A/m/t/]LFK. (SEQ ID NO:106). In some embodiments, the expression cassette comprises a heart and/or muscle-specific promoter, e.g., a MLC2v promoter or MCK promoter. In some embodiments, the coding sequence encodes SMN1, Frataxin, MTM1, GAA, TAZ, dystrophin, Mini dystrophin or factor IX.

In some embodiments, the disclosure provides an AAV vector comprising an AAV capsid comprising a targeting peptide as described herein, e.g., 5-7 amino acids of TVSALFK (SEQ ID NO:36), and an expression cassette comprising a coding sequence for protein of interest, a therapeutic transgene or non-coding RNA (e.g., as shown in Table B or Table 3), and at least one miR-208 targeting sequence as described herein. In some embodiments, the peptide insert comprises TVSALFK (SEQ ID NO:36). In some embodiments, the peptide insert comprises TVSALK (SEQ ID NO:32). In some embodiments, the peptide insert consists of TVSALFK (SEQ ID NO: 36). In some embodiments, the peptide insert consists of TVSALK (SEQ ID NO: 32). In some embodiments, the targeting peptides comprise a sequence of V[S/p][A/m/t/]L (SEQ ID NO:103), wherein the upper case letters are preferred at that position. In some embodiments, the targeting peptides comprise a sequence of TV[S/p][A/m/t/]L (SEQ ID NO:104). In some embodiments, the targeting peptides comprise a sequence of TV[S/p][A/m/t/]LK (SEQ ID NO:105). In some embodiments, the targeting peptides comprise a sequence of TV[S/p][A/m/t/]LFK. (SEQ ID NO:106). In some embodiments, the expression cassette comprises a heart and/or muscle-specific promoter, e.g., a MLC2v promoter or MCK promoter. In some embodiments, the coding sequence encodes SMN1, Frataxin, MTM1, GAA, TAZ, dystrophin, mini dystrophin or factor IX.

In some embodiments, the disclosure provides an AAV vector comprising an AAV capsid comprising a targeting peptide as described herein, e.g., 5-7 amino acids of TVSALFK (SEQ ID NO:36), and an expression cassette comprising a coding sequence for protein of interest as described herein, a therapeutic transgene or non-coding RNA (e.g., as shown in Table B or Table 3), and at least one miR-200C targeting sequence. In some embodiments, the peptide insert comprises TVSALFK (SEQ ID NO:36). In some embodiments, the peptide insert comprises TVSALK (SEQ ID NO:32). In some embodiments, the peptide insert consists of TVSALFK (SEQ ID NO: 36). In some embodiments, the peptide insert consists of TVSALK (SEQ ID NO: 32). In some embodiments, the targeting peptides comprise a sequence of V[S/p][A/m/t/]L (SEQ ID NO:103), wherein the upper case letters are preferred at that position. In some embodiments, the targeting peptides comprise a sequence of TV[S/p][A/m/t/]L (SEQ ID NO:104). In some embodiments, the targeting peptides comprise a sequence of TV[S/p][A/m/t/]LK (SEQ ID NO:105). In some embodiments, the targeting peptides comprise a sequence of TV[S/p][A/m/t/]LFK. (SEQ ID NO:106). In some embodiments, the expression cassette comprises a lung-specific promoter, e.g., a SP-B promoter or SP-C promoter. In some embodiments, the coding sequence encodes Alpha-1 antitrypsin or a therapeutic antibody, e.g., a SARS/COVID antibody.

For example, in some embodiments the cassette comprises a combination of the GFAP promoter and the microRNA122T. The GFAP promoter allows astrocyte-specific transgene expression in the CNS. This strategy diminishes expression of transgenes in the DRG cells and potentially avoids DRG toxicity that is reported to exhibit frequently after AAV-mediated CNS gene delivery. The microRNA122T at the 3′ UTR allows inhibition of expression in the liver since the expression of microRNA 122 is liver-specific. The present data shows that this liver-de-targeting element did not interfere with transgene expression in the CNS.

Therapeutic Proteins

In some embodiments, the AAV also includes a transgene sequence (i.e., a heterologous sequence), e.g., a transgene encoding a therapeutic agent, e.g., as described herein or as known in the art, or a reporter protein, e.g., a fluorescent protein, an enzyme that catalyzes a reaction yielding a detectable product, or a cell surface antigen. The transgene is preferably linked to regulatory sequences that promote/drive expression of the transgene in the target tissue.

Exemplary transgenes for use as therapeutics include toxins or suicide proteins, which are particularly useful in treating cancers.

Exemplary toxins include diphtheria toxin, tumor necrosis factor-related apoptosis-inducing ligand (TRAIL), or TNF-α.

Exemplary suicide proteins include herpes simplex virus thymidine kinase (HSVTK), bacterial or fungal cytosine deaminase (CD), carboxypeptidase G2 (CPG2), nitroreductase (NTR), Cytochrome P450 (CYP), purine nucleoside phosphorylase (PNP), horseradish peroxidase (HRP), or carboxylesterase (CE). Such suicide proteins can be used gene directed enzyme prodrug therapy (GDEPT), which combines passive, active, and transcriptional targeting strategies to provide anticancer activity. GDEPT is a two-step process whereby the cells are first transduced by a gene coding for a non-toxic enzyme (suicide gene) followed by administration of a non-toxic prodrug; see, e.g., Karjoo et al., Adv Drug Deliv Rev. 2016 Apr. 1; 99(Pt A): 113-128.

Other therapeutic transgenes include neuronal apoptosis inhibitory protein (NAIP), nerve growth factor (NGF), glial-derived growth factor (GDNF), brain-derived growth factor (BDNF), ciliary neurotrophic factor (CNTF), tyrosine hydroxlase (TH), GTP-cyclohydrolase (GTPCH), amino acid decorboxylase (AADC), aspartoacylase (ASPA), blood factors, such as β-globin, hemoglobin, tissue plasminogen activator, and coagulation factors; colony stimulating factors (CSF); interleukins, such as IL-1, IL-2, IL-3, IL-4, IL-5, IL-6, IL-7, IL-8, IL-9, etc.; growth factors, such as keratinocyte growth factor (KGF), stem cell factor (SCF), fibroblast growth factor (FGF, such as basic FGF and acidic FGF), hepatocyte growth factor (HGF), insulin-like growth factors (IGFs), bone morphogenetic protein (BMP), epidermal growth factor (EGF), growth differentiation factor-9 (GDF-9), hepatoma derived growth factor (HDGF), myostatin (GDF-8), nerve growth factor (NGF), neurotrophins, platelet-derived growth factor (PDGF), thrombopoietin (TPO), transforming growth factor alpha (TGF-α), transforming growth factor beta (TGF-β), and the like; soluble receptors, such as soluble TNF-α receptors, soluble VEGF receptors, soluble interleukin receptors (e.g., soluble IL-1 receptors and soluble type II IL-1 receptors), soluble gamma/delta T cell receptors, ligand-binding fragments of a soluble receptor, and the like; enzymes, such as α-glucosidase, imiglucarase, β-glucocerebrosidase, and alglucerase; enzyme activators, such as tissue plasminogen activator; chemokines, such as IP-10, monokine induced by interferon-gamma (Mig), Groa/IL-8, RANTES, MIP-1α, MIP-1β, MCP-1, PF-4, and the like; angiogenic agents, such as vascular endothelial growth factors (VEGFs, e.g., VEGF121, VEGF165, VEGF-C, VEGF-2), transforming growth factor-beta, basic fibroblast growth factor, glioma-derived growth factor, angiogenin, angiogenin-2; and the like; anti-angiogenic agents, such as a soluble VEGF receptor; protein vaccine; neuroactive peptides, such as nerve growth factor (NGF), bradykinin, cholecystokinin, gastin, secretin, oxytocin, gonadotropin-releasing hormone, beta-endorphin, enkephalin, substance P, somatostatin, prolactin, galanin, growth hormone-releasing hormone, bombesin, dynorphin, warfarin, neurotensin, motilin, thyrotropin, neuropeptide Y, luteinizing hormone, calcitonin, insulin, glucagons, vasopressin, angiotensin II, thyrotropin-releasing hormone, vasoactive intestinal peptide, a sleep peptide, and the like; thrombolytic agents; atrial natriuretic peptide; relaxin; glial fibrillary acidic protein; follicle stimulating hormone (FSH); human alpha-1 antitryp sin; leukemia inhibitory factor (LIF); transforming growth factors (TGFs); tissue factors, luteinizing hormone; macrophage activating factors; tumor necrosis factor (TNF); neutrophil chemotactic factor (NCF); nerve growth factor; tissue inhibitors of metalloproteinases; vasoactive intestinal peptide; angiogenin; angiotropin; fibrin; hirudin; IL-1 receptor antagonists; and the like. Some other examples of protein of interest include ciliary neurotrophic factor (CNTF); neurotrophins 3 and 4/5 (NT-3 and 4/5); glial cell derived neurotrophic factor (GDNF); aromatic amino acid decarboxylase (AADC); hemophilia related clotting proteins, such as Factor VIII, Factor IX, Factor X; dystrophin or nini-dystrophin; lysosomal acid lipase; phenylalanine hydroxylase (PAH); glycogen storage disease-related enzymes, such as glucose-6-phosphatase, acid maltase, glycogen debranching enzyme, muscle glycogen phosphorylase, liver glycogen phosphorylase, muscle phosphofructokinase, phosphorylase kinase (e.g., PHKA2), glucose transporter (e.g., GLUT2), aldolase A, β-enolase, and glycogen synthase; lysosomal enzymes (e.g., beta-N-acetylhexosaminidase A); and any variants thereof.

The transgene can encode an antibody, e.g., an immune checkpoint inhibitory antibody, e.g., to PD-L1, PD-1, CTLA-4 (Cytotoxic T-Lymphocyte-Associated Protein-4; CD152); LAG-3 (Lymphocyte Activation Gene 3; CD223); TIM-3 (T-cell Immunoglobulin domain and Mucin domain 3; HAVCR2); TIGIT (T-cell Immunoreceptor with Ig and ITIM domains); B7-H3 (CD276); VSIR (V-set immunoregulatory receptor, aka VISTA, B7H5, C10orf54); BTLA 30 (B- and T-Lymphocyte Attenuator, CD272); GARP (Glycoprotein A Repetitions; Predominant; PVRIG (PVR related immunoglobulin domain containing); or VTCN1 (Vset domain containing T cell activation inhibitor 1, aka B7-H4).

Other transgenes can include small or inhibitory nucleic acids that alter/reduce expression of a target gene, e.g., siRNA, shRNA, miRNA, antisense oligos, or long non-coding RNAs that alter gene expression (see, e.g., WO2012087983 and US20140142160), or CRISPR Cas9/cas12a and guide RNAs.

Methods of Use

The methods and compositions described herein can be used to express a transgene in a tissue-specific manner, e.g., to the central nervous system (brain), heart, muscle, or dorsal root ganglion or spinal cord (peripheral nervous system). In some embodiments, the methods include systemic delivery of the compositions described herein. In some embodiments, the methods include delivery to specific brain regions, e.g., cortex, cerebellum, hippocampus, substantia nigra, or amygdala. In some embodiments, the methods include delivery to neurons, astrocytes, and/or glial cells.

In some embodiments, the methods and compositions, e.g., AAVs, are used to deliver a nucleic acid sequence to a subject who has a disease, e.g., a disease of the CNS; see, e.g., U.S. Pat. Nos. 9,102,949; 9,585,971; and US20170166926. In some embodiments, the subject has a condition listed in Table B; in some embodiments, the vectors are used to deliver a therapeutic agent listed in Table B for treating the corresponding disease listed in Table B. The therapeutic agent can be delivered as a nucleic acid, e.g. via a viral vector, wherein the nucleic acid encodes a therapeutic protein or other nucleic acid such as an antisense oligo, siRNA, shRNA, and so on; or as a fusion protein/complex with a targeting peptide as described herein.

TABLE B
Diseases

| Examples of diseases | Tissue targeted | Therapeutic agent |
|---|---|---|
| Parkinson's disease | CNS | GDNF, AADC |
| Alzheimer's disease | CNS | Tau antibody, APP |
| | | antibody |
| Huntington's disease | CNS | miRNA targeting HTT |
| Amyotrophic lateral | CNS | shRNA targeting SOD |
| sclerosis | | |
| Multiple sclerosis | CNS | IFN-beta |
| Epilepsy | CNS | Neuropeptide Y |
| Stroke | CNS | IGF-1, osteopontin |
| Brain cancer | CNS | HSV.TK1, PD-1/PD- |
| | | L1 antibody |
| Spinocerebellar | CNS | RNAi targeting |
| ataxia | | ataxin |
| Canavan disease | CNS | ASPA |
| Metachromatic | Nervous systems | ARSA, PSAP |
| leukodystrophy | | |
| Spinal muscular | Neuromuscular system | SMN1 |
| atrophy | | |
| Friedreich's ataxia | Nervous systems, | Frataxin |
| | heart | |
| X-linked myotubular | Neuromuscular system | MTM1 |
| myopathy | | |
| Pompe disease | Lysosome (global | GAA |
| | including CNS) | |
| Barth syndrome | Heart, muscle | TAZ |
| Duchenne muscular | Muscle | dystrophin |
| dystrophy | | |
| Wilson's disease | Brain, liver | ATP7B |
| Crigler-Najjar | Liver | UGT1A1 |
| syndrome type 1 | | |

In some embodiments, the methods and compositions, e.g., AAVs, are used to deliver a nucleic acid sequence encoding a transgene that includes a toxin or suicide gene to a subject who has brain cancer. Brain cancers include gliomas (e.g., glioblastoma multiforme (GBM)), metastases (e.g., from lung, breast, melanoma, or colon cancer), meningiomas, pituitary adenomas, and acoustic neuromas. Thus the methods can include systemically, e.g., intravenously, administering an AAV.CPP.16 as described herein, encoding a toxin or suicide protein to a subject who has been diagnosed with brain cancer. In some embodiments, where the therapeutic protein is a suicide protein, the methods further include contact administering a nontoxic prodrug that is a substrate for the suicide protein, wherein action of the suicide protein on the nontoxic prodrug results in production of a toxic metabolite that induces cell death. Examples include wherein the suicide protein is herpes simplex virus thymidine kinase (HSVTK) and the nontoxic prodrug is ganciclovir (GCV); the suicide protein is cytosine deaminase (CD) and the nontoxic prodrug is 5-flourouracil (5-FU); the suicide protein is carboxypeptidase G2 (CPG2) and the nontoxic prodrug is nitrogen mustard (NM) or a derivate thereof such as ZD2767P or CMDA (4-[(2-chloroethyl)(2-mesyloxyethyl)amino]benzoyl-L-glutamicacid); the suicide protein is nitroreductase (NTR) and the nontoxic prodrug is CB1954 or an analog thereof; the suicide protein is Cytochrome P450 (CYP) and the nontoxic prodrug is an oxazaphosphorine drug such as cyclophosphamide (CPA) and ifosfomide (IFO); the suicide protein is purine nucleoside phosphorylase (PNP) and the nontoxic prodrug is 6-Methylpurine Deoxyriboside or an analog thereof, e.g., fludarabine phosphate (F-araAMP) or 2-fluoro-2-deoxyadenosine (F-dAdo); the suicide gene is horseradish peroxidase (HRP) and the nontoxic prodrug is indole-3-acetic acid (HRP/IAA); or the suicide protein is carboxylesterase (CE) and the nontoxic prodrug is irinotecan. See, e.g., Karjoo et al., Adv Drug Deliv Rev. 2016 Apr. 1; 99(Pt A): 113-128.

In some embodiments, the AAV is targeted to glial cells, e.g., cancerous glial cells in a subject, e.g., the cancer is glioblastoma.

In some embodiments, the methods also include co-administering a chemotherapeutic agent. In some embodiments, the chemotherapeutic agent is a toxin or cytotoxic drug, including but not limited to temozolamide, lomustine, or a combination thereof. See, e.g., Herrlinger et al., Lancet. 2019 Feb. 16;393(10172):678-688. The methods can also include administering radiation, surgical resection, or both.

Pharmaceutical Compositions and Methods of Administration

The methods described herein include the use of pharmaceutical compositions comprising or consisting of AAVs as described herein an active ingredient.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration.

Pharmaceutical compositions are typically formulated to be compatible with its intended route of administration. Examples of routes of administration include parenteral, e.g., intravenous, intraarterial, subcutaneous, intraperitoneal intramuscular or injection or infusion administration. Delivery can thus be systemic or localized.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, N.Y.). For example, solutions or suspensions used for parenteral application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a kit, container, pack, or dispenser together with instructions for administration.

### EXAMPLES

The invention is further described in the following examples, which do not limit the scope of the invention described in the claims.

### Example 1. Design of Tissue Selective AAV Vectors

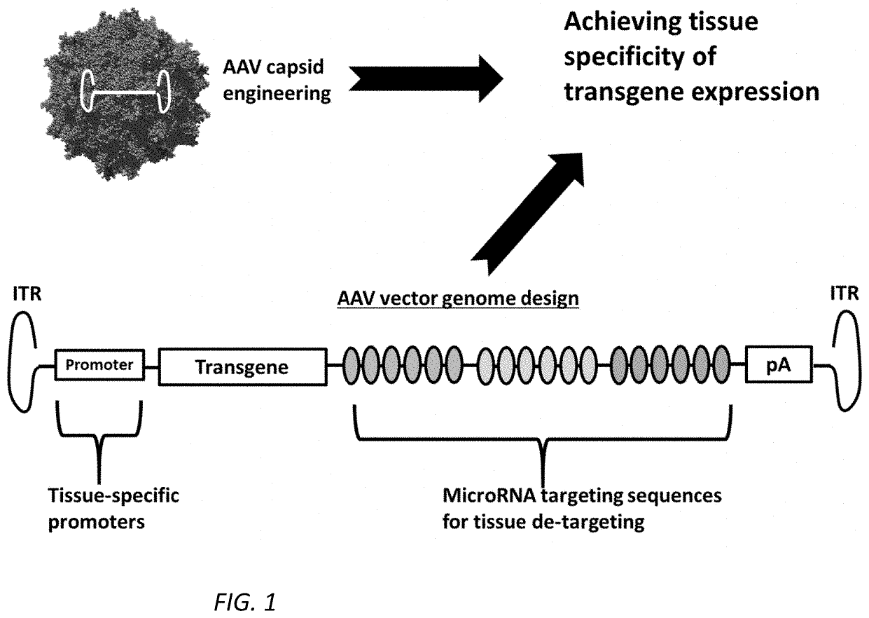

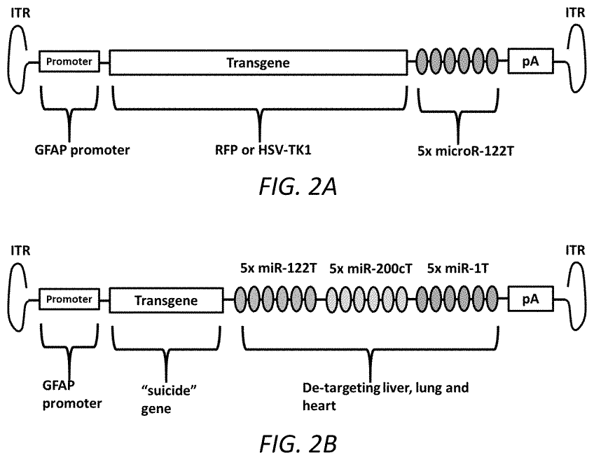

As illustrated in FIG. 1, selective targeting of transgenes for gene therapy depends on delivery vectors such as AAVs that determine the tissue and cell types where the expression cassettes are delivered and the expression cassettes themselves that determine how the transgenes are expressed inside the target cells. Vector properties sometimes modulate the regulatory elements in expression cassettes. For instance, it is reported AAV capsid could affect the tissue specificity of promoter function. Thus, a holistic approach is required to design optimal gene therapy vector with tissue selectivity. For instance, for CNS gene therapy, AAV.CPP.16 can be used as a delivery vector given its known CNS tropism after both systemic and intrathecal administration. Regulatory elements contained in expression cassettes can include CNS selective promoters, specific translation regulatory sequences, and 3′ UTR elements. Given the known tissue specific expression of many microRNAs such as microRNA 122 in the liver, microRNA 124a in the CNS, microRNA 1 in the heart and muscle and microRNA 200c in the lung, arrays of microRNA targeting sequences can be properly designed and incorporated into the expression cassette (FIG. 2B). Binding of microRNA to its corresponding targeting sequence can inhibit the expression of the transgene in a tissue specific manner, resulting in selective tissue detargeting.

### Example 2. A Liver-Detargeting, CNS-Tropic Vector Based on AAV.CPP.16

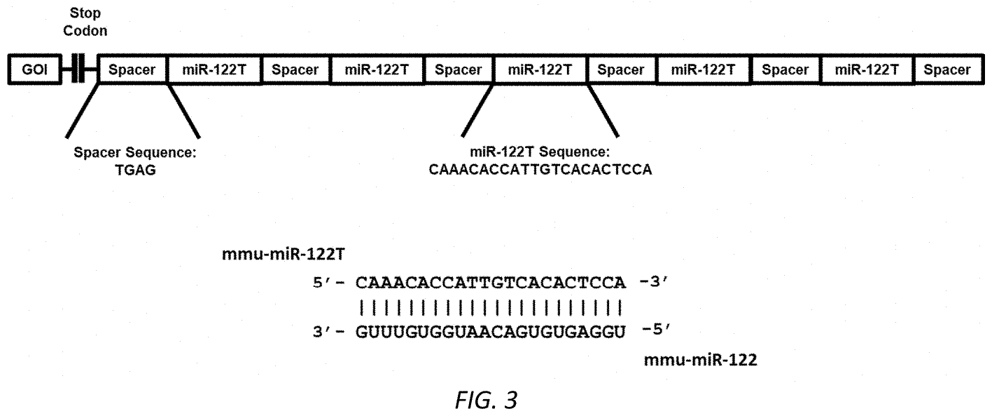

To demonstrate of proof-of-concept, a liver-detargeting, AAV.CPP.16-based vector was developed for CNS cancer gene therapy. As illustrated in FIG. 2A, flanked by two inverted terminal repeats (ITRs) is an AAV expression cassette containing a GFAP promoter which is active in astrocytes and some glioma cells, transgene which is RFP reporter or HSV-TK1 suicide gene, an array of 5 repeats of microRNA 122 targeting sequences separated by design spacers, followed by a polyadenylation sequence. More details of the microRNA 122T array was illustrated in FIG. 3.

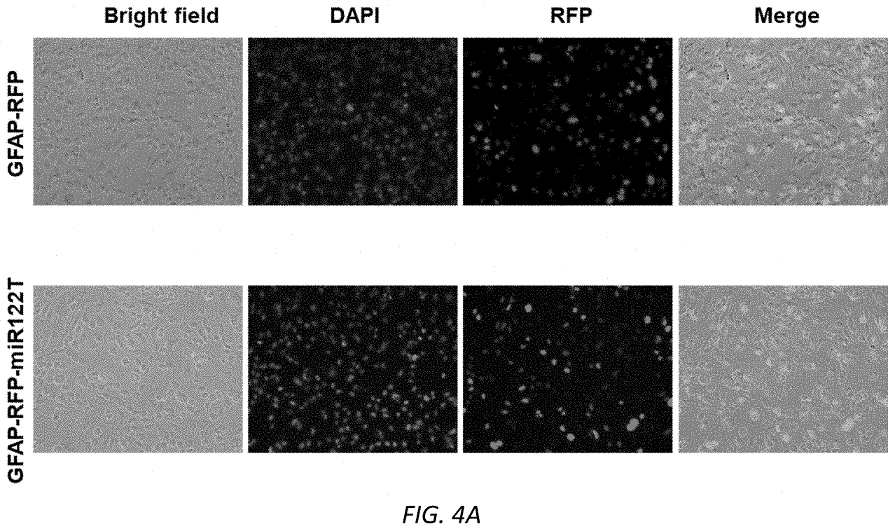

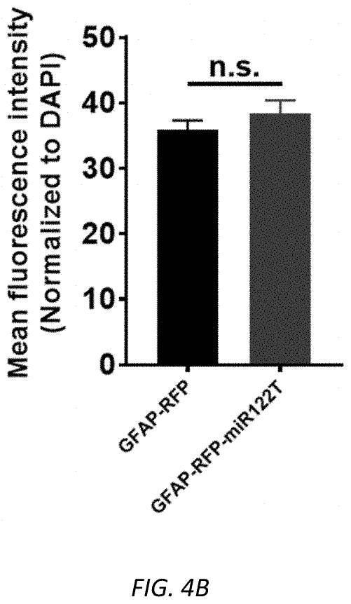

To demonstrate the function of such expression cassette, AAVs.CPP.16 containing such cassette (AAV.CPP.16-GFAP-RFP-miR122T) were produced along with AAV.CPP.16-GFAP-RFP. As shown in FIG. 4A, both vectors were able to transduce glioma cell line GL261 cells in vitro. No significant difference of transduction efficiency was observed, as quantified in FIG. 4B, suggesting miR122T array does not interfere with transgene expression in such glioma cells.

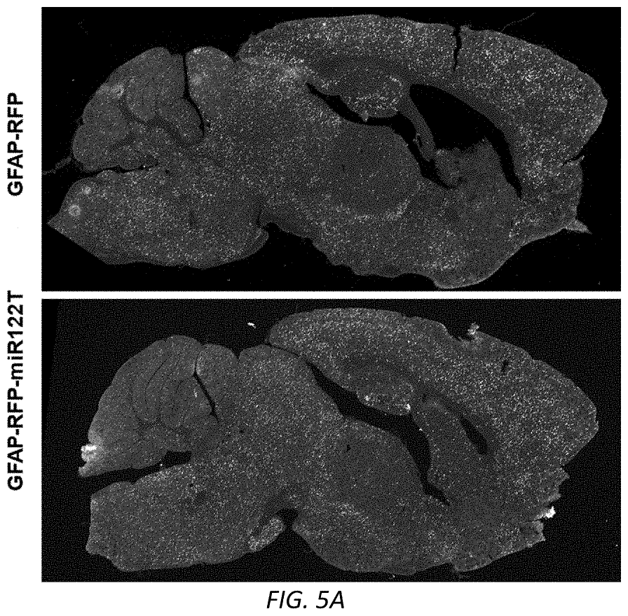

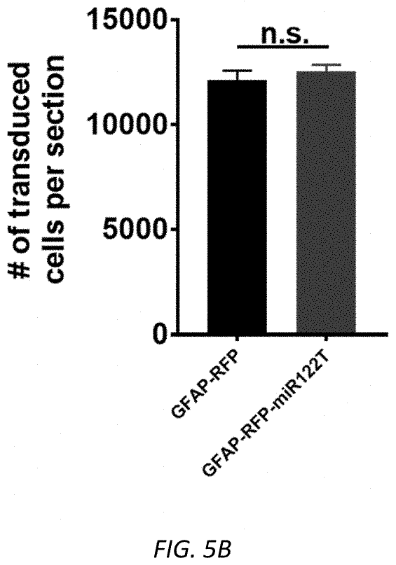

To further test both vectors in vivo, AAVs.CPP.16-GFAP-RFP-miR122T and AAVs.CPP.16-GFAP-RFP were intravenously injected into adult BALB/c mice at a dose of 1e12 vg per animal. 3 weeks later, animals were processed and brains, as well as other peripheral tissues, were examined for RFP expression. As shown in FIG. 5A, RFP expression was observed across the entire brain sections (show in white), and no significant difference of transduction efficiency was observed as quantified in FIG. 5B, suggesting miR122T array does not interfere transgene expression in the CNS in vivo.

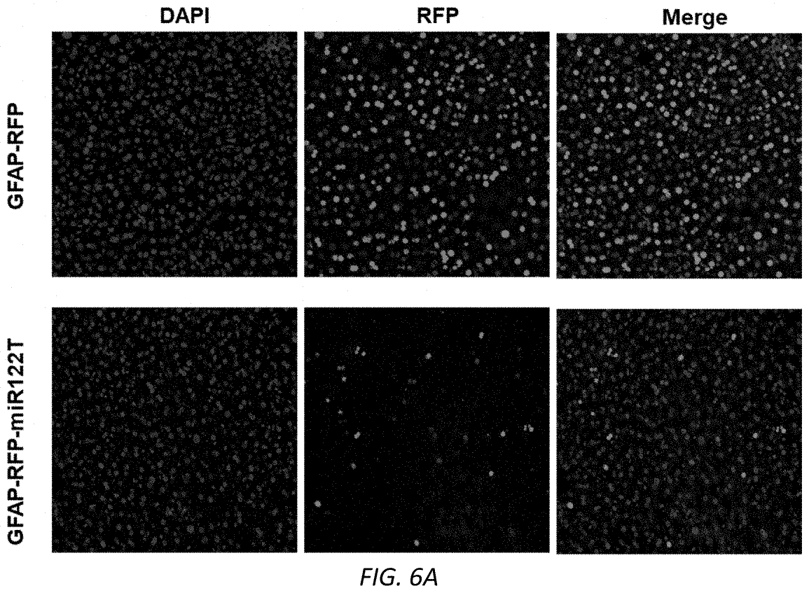

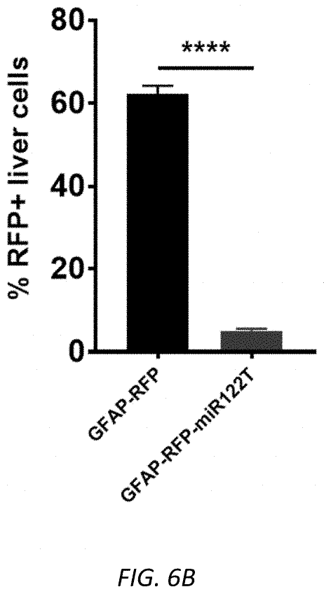

Significant difference of transduction was found in the liver between the two AAV vectors. As shown in FIG. 6A, 1e12 vg AAV.CPP.16-GFAP-RFP resulted in a high percentage of liver cells transduced after IV delivery, while incorporation of the miR122T array significantly diminished RFP expression in the liver, as quantified in FIG. 6B.

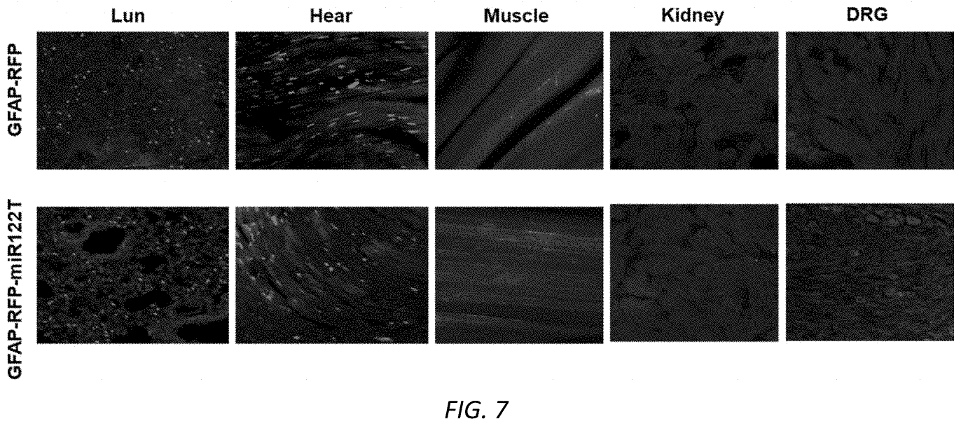

As shown in FIG. 7, both AAV.CPP.16-GFAP-RFP and AAV.CPP.16-GFAP-RFP-miR122T transduce the lung and the heart with comparable efficiency. Little transduction was observed in the skeletal muscle, kidney and the dorsal root ganglion (DRG).

### Example 3. A Liver-Detargeting AAV Gene Therapy Against Glioblastoma

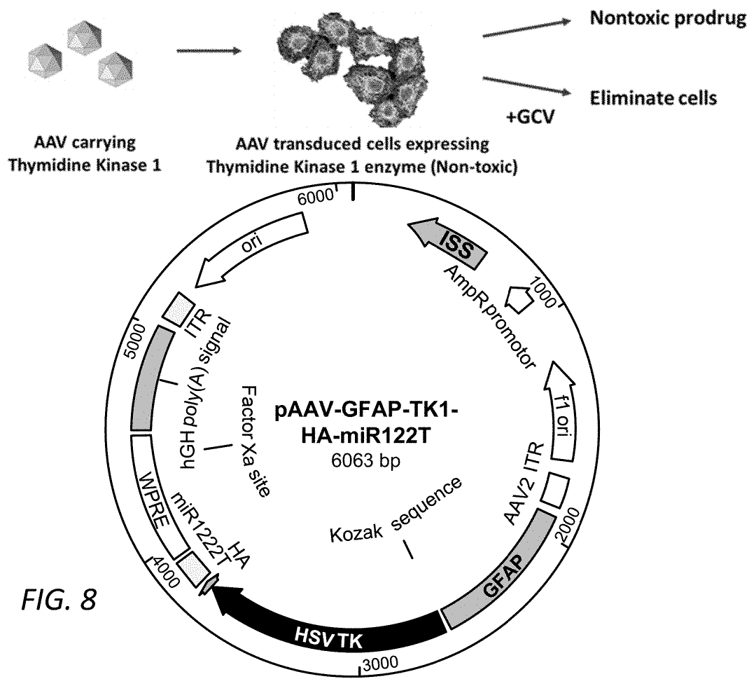

Herpes simplex virus thymidine kinase 1 (i.e., HSV TK1) is a “suicide” gene that can be applied for killing tumor cells. As illustrated in FIG. 8, after HSV TK1 is expressed in target cells by AAV, HSV TK1 can turn the nontoxic prodrug ganciclovir (GCV) into a cytotoxic drug.

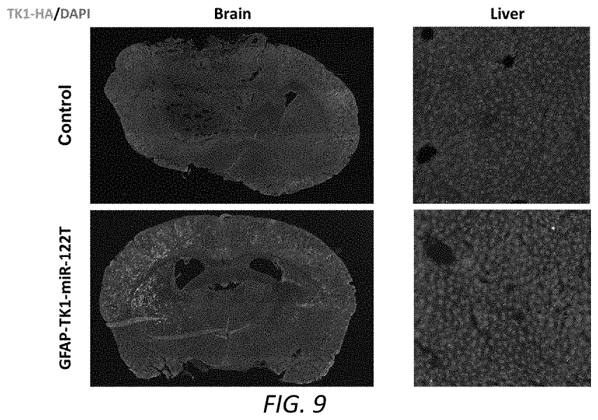

It was found that when AAVs.CPP.16-GFAP-TK1-miR122T were intravenously administered into adult mice bearing GL261 tumor cells, TK1 expression was observed in the brain while no such expression was observed in the liver, as shown in FIG. 9.

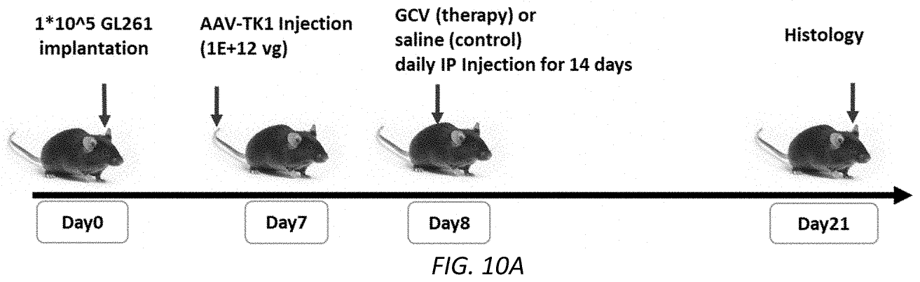

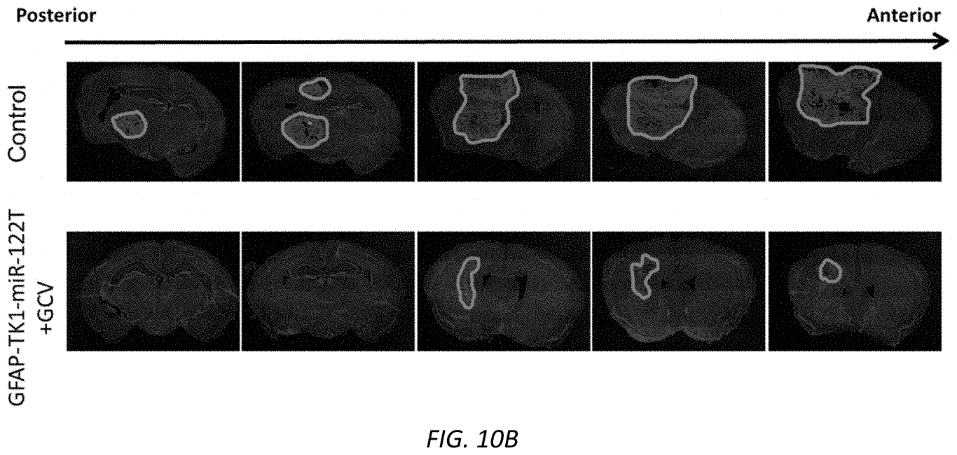

It was further found that combining AAVs.CPP.16-GFAP-TK1-miR122T with GCV administration dramatically reduced the glioma tumor size in the brain compared with shame control treatment, as shown in FIG. 10. Such gene therapy extended the survival of tumor-bearing mice. No liver toxicity was observed. As previous research revealed the liver toxicity as a major obstacle for developing systemic suicide gene therapy, our new vector provides a solution to circumvent such obstacle.

### Example 4. AAV.CPP.16-Mediated HSV-TK1 Gene Therapy Prolongs Survival in a Glioblastoma Model

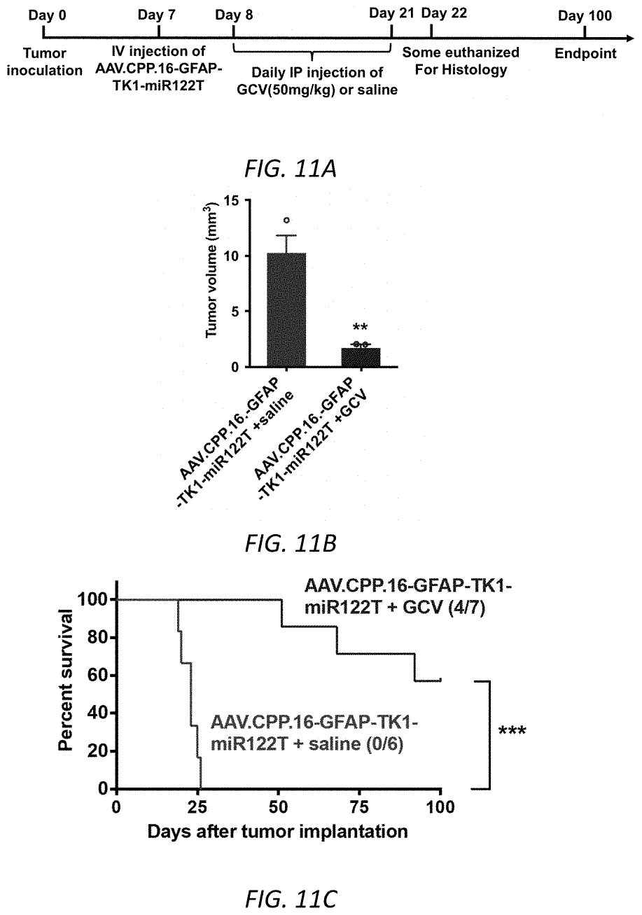

In this example, AAV.CPP.16-GFAP-TK1-miR122T was intravenously administered to GL261 glioblastoma tumor-bearing mice using the experimental timeline depicted in FIG. 11A. Animals were inoculated with GL261 tumor cells at day 0. AAV.CPP.16-GFAP-TK1-miR122T was intravenously administered at day 7 after tumor cell inoculation. Beginning at day 8, ganciclovir (GCV1 50 mg/kg, treatment group) or saline (control group) was administered intraperitoneally daily for 14 days. On day 22, three treatment group animals and three control group animals were sacrificed for histological studies of tumor volume. Remaining animals (n=6-7 for each of treatment and control group) were observed and survival outcomes were recorded through day 100. As shown in FIG. 11B, AAV.CPP.16-GFAP-TK1-miR122T plus ganciclovir significantly (p<0.01, Student's t-test, two tails) reduced tumor sizes after two weeks of treatment following administration of AAV.CPP.16-GFAP-TK1-miR122T, relative to the control group. As shown in FIG. 11C, AAV.CPP.16-GFAP-TK1-miR122T plus ganciclovir significantly (p<0.001, log-rank test) prolonged survival of treatment group animals relative to control group animals observed for 100 days. Taken together, these results indicate therapeutic efficacy of AAV.CPP.16-mediated HSV-TK1 suicide gene therapy in a mouse model of glioblastoma.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An adeno-associated virus (AAV) vector, preferably comprising a target tissue-tropic capsid, optionally AAV.CPP.16, AAV.CPP.21 or AAV9, and further comprising an expression cassette comprising, preferably from 5′ to 3′:
a promoter that drives expression in cells of the target tissue,
an optional secretory signal peptide sequence,
a coding sequence for a protein of interest,
an optional Woodchuck Hepatitis Virus Posttranscriptional Regulatory Element (WPRE),
a polyA signal sequence, and
at least one microRNA targeting sequence selected from a microRNA 122 targeting sequence (miR-122T), optionally targeting the 5p strand, optionally comprising CAAACACCATTGTCACACTCCA (SEQ ID NO: 21); a microRNA 124 targeting sequence (miR-124T), optionally targeting the 5p strand, optionally comprising ATCAAGGTCCGCTGTGAACACG (SEQ ID NO: 20); a microRNA 200c targeting sequence (miR-200cT), optionally targeting the 5p strand, optionally CCAAACACTGCTGGGTAAGACG (SEQ ID NO: 22); and/or microRNA 1 targeting sequence (miR-1T), optionally targeting the 5p strand, optionally ATGGGCATATAAAGAAGTATGT (SEQ ID NO: 24), at the 3′ UTR.

2. The AAV vector of claim 1, wherein the capsid is CNS tropic, optionally neuronal or glial-tropic.

3. The AAV vector of claim 2, wherein the promoter that drives expression in the CNS drives expression in neuronal cells or glial cells.

4. The AAV vector of claim 3, wherein the promoter that drives expression in glial cells is a GFAP promoter, gfaABC1D promoter, gfa2 promoter, ALDH1L1 promoter, SLC1A3 promoter, Gjb6 promoter, Mbp promoter, MAG promoter, CBh promoter, F4/80 promoter, CD68 promoter, or CD11B promoter.

5. The AAV vector of claim 3, wherein the promoter that drives expression in neuronal cells is a neuronal-specific enolase (NSE) promoter, Synapsin promoter, calcium/calmodulin-dependent protein kinase II promoter, tubulin alpha 1 promoter, platelet-derived growth factor beta chain promoter, parvalbumin promoter, GAD67 promoter or CCK promoter.

6. The AAV vector of claim 1, wherein the promoter is a ubiquitous promoter, optionally major immediate early human cytomegalovirus promoter (MIEhCMV), Chicken (3-Actin Promoter (CBA); Human Cytomegalovirus Immediate/Early Gene Promoter and Enhancer (CMV); Chicken 3-Actin/Cytomegalovirus Hybrid Promoter (CAG); Rous Sarcoma Virus Long Terminal Repeat Promoter (RSV); SV40 promoter; EF 1 alpha promoter.

7. The AAV vector of claim 1, further comprising an enhancer, optionally CMV-Enhancer, mD1x enhancer, AQP4 enhancer.

8. The AAV vector of claim 1, wherein the optional secretory signal peptide sequence is a Human IL-2 signal peptide (optionally MYRMQLLSCIALSLALVTNS, SEQ ID NO: 16), human albumin signal peptide, human alpha 1-antitrypsin signal peptide, or human factor VIII signal peptide.

9. The AAV vector of claim 1, wherein the polyA signal sequence is from human growth hormone (hGH), SV40, bovine growth hormone (bGH), or beta-globin, optionally rabbit beta-globin (rbGlob).

10. The AAV vector of claim 1, comprising a plurality of, optionally 2-10 or 3-5, microRNA targeting sequences, optionally separated by spacer sequence (optionally of 1-50 nucleotides).

11. The AAV vector of claim 1, wherein the protein of interest is a therapeutic protein.

12. The AAV vector of claim 11, wherein the therapeutic protein of interest is an antibody such as immune checkpoint inhibitors.

13. The AAV vector of claim 11, wherein the therapeutic protein of interest is a toxin, a suicide protein, or an antibody.

14. The AAV vector of claim 13, wherein the toxin is diphtheria toxin, tumor necrosis factor-related apoptosis-inducing ligand (TRAIL), or TNF-α.

15. The AAV vector of claim 14, wherein the suicide protein is herpes simplex virus thymidine kinase (HSVTK), bacterial or fungal cytosine deaminase (CD), carboxypeptidase G2 (CPG2), nitroreductase (NTR), Cytochrome P450 (CYP), purine nucleoside phosphorylase (PNP), horseradish peroxidase (HRP), or carboxylesterase (CE).

16. The AAV Vector of claim 1, which targets the CNS; wherein the capsid is AAV.CPP.16 or AAV.CPP.21; the promoter is a GFAP promoter or a Syn promoter; and the microRNA targeting sequences target one, two, or all three of microRNA 122, microRNA 200c and microRNA 1.

17. The AAV vector of claim 16, wherein the protein of interest is MeCP2, CNTF, or NGF.

18. The AAV Vector of claim 1, which targets the liver; wherein the capsid is AAV.CPP.16 or AAV9; the promoter is a LSP promoter or an α1-antitrypsin promoter; the microRNA targeting sequences target one, two, or all three of microRNA 124, microRNA 200c and microRNA 1.

19. The AAV vector of claim 18, wherein the protein of interest is Factor VIII or factor IX.

20. The AAV Vector of claim 1, which targets the heart or other muscle; wherein the capsid is AAV.CPP.16; the promoter is a MLC2v promoter or MCK promoter; and the microRNA targeting sequences target one, two, or all three of microRNA 122, microRNA 200c and microRNA 124.

21. The AAV vector of claim 20, wherein the protein of interest is mini dystrophin or factor IX.

22. The AAV Vector of claim 1, which targets the lung, wherein the capsid is AAV.CPP.16; the promoter is an SP-B promoter or SP-C promoter; and the microRNA targeting sequences target one, two, or all three of microRNA 122, microRNA 124 and microRNA 1.

23. The AAV vector of claim 22, wherein the protein of interest is Alpha-1 antitrypsin or an anti-SARS antibody.

24. A method of directing expression of a protein of interest in a cell in a target tissue, preferably without substantial expression of the protein of interest in non-target cells, the method comprising introducing the AAV vector of claim 1 into the cell.

25. A method of inducing cell death in cell in a glial cell or a neuronal cell, the method comprising introducing the AAV vector of claim 1 into the cell.

26. The method of claim 25, wherein the therapeutic protein of interest is a suicide protein, and the method further comprises contact the cell with a nontoxic prodrug that is a substrate for the suicide protein, wherein action of the suicide protein on the nontoxic prodrug results in production of a toxic metabolite that induces cell death.

27. The method of claim 26, wherein the suicide protein is herpes simplex virus thymidine kinase (HSVTK) and the nontoxic prodrug is ganciclovir (GCV); the suicide protein is cytosine deaminase (CD) and the nontoxic prodrug is 5-flourouracil (5-FU); the suicide protein is carboxypeptidase G2 (CPG2) and the nontoxic prodrug is nitrogen mustard (NM) or a derivate thereof such as ZD2767P or CMDA (4-[(2-chloroethyl)(2-mesyloxyethyl)amino]benzoyl-L-glutamicacid); the suicide protein is nitroreductase (NTR) and the nontoxic prodrug is CB1954 or an analog thereof; the suicide protein is Cytochrome P450 (CYP) and the nontoxic prodrug is an oxazaphosphorine drug such as cyclophosphamide (CPA) and ifosfomide (IFO); the suicide protein is purine nucleoside phosphorylase (PNP) and the nontoxic prodrug is 6-Methylpurine Deoxyriboside or an analog thereof, optionally fludarabine phosphate (F-araAMP) or 2-fluoro-2-deoxyadenosine (F-dAdo); the suicide gene is horseradish peroxidase (HRP) and the nontoxic prodrug is indole-3-acetic acid (HRP/IAA); or the suicide protein is carboxylesterase (CE) and the nontoxic prodrug is irinotecan.

28. The method of claim 25, wherein the glial cell is a cancer cell in a subject.

29. The method of claim 28, wherein the cancer is glioblastoma.


SEQUENCE LISTING (115 sequences)

SEQ ID NO: 1 - protein, 736 aa, Artificial Sequence
  Description of Artificial Sequence Synthetic polypeptide
  AAV9 viral vector
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 2 - protein, 742 aa, Artificial Sequence
  Description of Artificial Sequence Synthetic polypeptide
  AAV9.CPP16 viral vector
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQTVSALKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 3 - protein, 743 aa, Artificial Sequence
  Description of Artificial Sequence Synthetic polypeptide
  AAV9.CPP21 viral vector
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQTVSALFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 4 - DNA, 479 bp, Homo sapiens
acgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 5 - DNA, 135 bp, Artificial Sequence
  Description of Artificial Sequence Synthetic polynucleotide
aacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 6 - DNA, 228 bp, Bos taurus
cgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 7 - DNA, 527 bp, Oryctolagus cuniculus
ttcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatc

SEQ ID NO: 8 - protein, 25 aa, Homo sapiens
MGVLLTQRTLLSLVLALLFPSMASM

SEQ ID NO: 9 - protein, 19 aa, Homo sapiens
MGWSCIILFLVATATGVHS

SEQ ID NO: 10 - protein, 21 aa, Homo sapiens
MWWRLWWLLLLLLLLWPMVWA

SEQ ID NO: 11 - protein, 22 aa, Homo sapiens
MDMRVPAQLLGLLLLWLRGARC

SEQ ID NO: 12 - protein, 16 aa, Homo sapiens
MPLLLLLPLLWAGALA

SEQ ID NO: 13 - protein, 23 aa, Homo sapiens
MDAMKRGLCCVLLLCGAVFVSPS

SEQ ID NO: 14 - protein, 18 aa, Homo sapiens
MAFLWLLSCWALLGTTFG

SEQ ID NO: 15 - protein, 15 aa, Homo sapiens
MNLLLILTFVAAAVA

SEQ ID NO: 16 - protein, 20 aa, Homo sapiens
MYRMQLLSCIALSLALVTNS

SEQ ID NO: 17 - protein, 16 aa, Homo sapiens
MKWVTFISLLFSSAYS

SEQ ID NO: 18 - protein, 24 aa, Homo sapiens
MALWMRLLPLLALLALWGPDPAAA

SEQ ID NO: 19 - protein, 24 aa, Homo sapiens
MPSSVSWGILLLAGLCCLVPVSLA

SEQ ID NO: 20 - DNA, 22 bp, Homo sapiens
atcaaggtccgctgtgaacacg

SEQ ID NO: 21 - DNA, 22 bp, Homo sapiens
caaacaccattgtcacactcca

SEQ ID NO: 22 - DNA, 22 bp, Homo sapiens
ccaaacactgctgggtaagacg

SEQ ID NO: 23 - DNA, 23 bp, Mus musculus
tatgggcatataaagaagtatgt

SEQ ID NO: 24 - DNA, 22 bp, Homo sapiens
atgggcatataaagaagtatgt

SEQ ID NO: 25 - RNA, 22 nt, Homo sapiens
cguguucacagcggaccuugau

SEQ ID NO: 26 - RNA, 22 nt, Homo sapiens
uggagugugacaaugguguuug

SEQ ID NO: 27 - RNA, 22 nt, Homo sapiens
cgucuuacccagcaguguuugg

SEQ ID NO: 28 - RNA, 23 nt, Mus musculus
acauacuucuuuauaugcccaua

SEQ ID NO: 29 - protein, 5 aa, Artificial Sequence
  Description of Artificial Sequence Synthetic peptide
VPALR

SEQ ID NO: 30 - protein, 5 aa, Artificial Sequence
  Description of Artificial Sequence Synthetic peptide
VSALK

SEQ ID NO: 31 - protein, 6 aa, Artificial Sequence
  Description of Artificial Sequence Synthetic peptide
TVPALR

SEQ ID NO: 32 - protein, 6 aa, Artificial Sequence
  Description of Artificial Sequence Synthetic peptide
TVSALK

SEQ ID NO: 33 - protein, 7 aa, Artificial Sequence
  Description of Artificial Sequence Synthetic peptide
FTVSALK

SEQ ID NO: 34 - protein, 7 aa, Artificial Sequence
  Description of Artificial Sequence Synthetic peptide
LTVSALK

SEQ ID NO: 35 - protein, 7 aa, Artificial Sequence
  Description of Artificial Sequence Synthetic peptide
TFVSALK

SEQ ID NO: 36 - protein, 7 aa, Artificial Sequence
  Description of Artificial Sequence Synthetic peptide
TVSALFK

SEQ ID NO: 37 - protein, 7 aa, Artificial Sequence
  Description of Artificial Sequence Synthetic peptide
TVPALFR

SEQ ID NO: 38 - protein, 7 aa, Artificial Sequence
  Description of Artificial Sequence Synthetic peptide
TVPMLFK

SEQ ID NO: 39 - protein, 7 aa, Artificial Sequence
  Description of Artificial Sequence Synthetic peptide
TVPTLFK

SEQ ID NO: 40 - protein, 6 aa, Artificial Sequence
  Description of Artificial Sequence Synthetic peptide
TVPMLK

SEQ ID NO: 41 - protein, 6 aa, Artificial Sequence
  Description of Artificial Sequence Synthetic peptide
TVPTLK

SEQ ID NO: 42 - protein, 5 aa, Artificial Sequence
  Description of Artificial Sequence Synthetic peptide
VPMLK

SEQ ID NO: 43 - protein, 5 aa, Artificial Sequence
  Description of Artificial Sequence Synthetic peptide
VPTLK

SEQ ID NO: 44 - protein, 6 aa, Artificial Sequence
  Description of Artificial Sequence Synthetic peptide
VPMLKE

SEQ ID NO: 45 - protein, 6 aa, Artificial Sequence
  Description of Artificial Sequence Synthetic peptide
VPTLKD

SEQ ID NO: 46 - protein, 6 aa, Artificial Sequence
  Description of Artificial Sequence Synthetic peptide
VPALRD

SEQ ID NO: 47 - protein, 6 aa, Artificial Sequence
  Description of Artificial Sequence Synthetic peptide
VSALKE

SEQ ID NO: 48 - protein, 6 aa, Artificial Sequence
  Description of Artificial Sequence Synthetic peptide
VSALKD

SEQ ID NO: 49 - protein, 6 aa, Artificial Sequence
  Description of Artificial Sequence Synthetic peptide
TAVSLK

SEQ ID NO: 50 - protein, 6 aa, Artificial Sequence
  Description of Artificial Sequence Synthetic peptide
TALVSK

SEQ ID NO: 51 - protein, 6 aa, Artificial Sequence
  Description of Artificial Sequence Synthetic peptide
TVLSAK

SEQ ID NO: 52 - protein, 6 aa, Artificial Sequence
  Description of Artificial Sequence Synthetic peptide
TLVSAK

SEQ ID NO: 53 - protein, 6 aa, Artificial Sequence
  Description of Artificial Sequence Synthetic peptide
TMVPLK

SEQ ID NO: 54 - protein, 6 aa, Artificial Sequence
  Description of Artificial Sequence Synthetic peptide
TMLVPK

SEQ ID NO: 55 - protein, 6 aa, Artificial Sequence
  Description of Artificial Sequence Synthetic peptide
TVLPMK

SEQ ID NO: 56 - protein, 6 aa, Artificial Sequence
  Description of Artificial Sequence Synthetic peptide
TLVPMK

SEQ ID NO: 57 - protein, 6 aa, Artificial Sequence
  Description of Artificial Sequence Synthetic peptide
TTVPLK

SEQ ID NO: 58 - protein, 6 aa, Artificial Sequence
  Description of Artificial Sequence Synthetic peptide
TTLVPK

SEQ ID NO: 59 - protein, 6 aa, Artificial Sequence
  Description of Artificial Sequence Synthetic peptide
TVLPTK

SEQ ID NO: 60 - protein, 6 aa, Artificial Sequence
  Description of Artificial Sequence Synthetic peptide
TLVPTK

SEQ ID NO: 61 - protein, 6 aa, Artificial Sequence
  Description of Artificial Sequence Synthetic peptide
TAVPLR

SEQ ID NO: 62 - protein, 6 aa, Artificial Sequence
  Description of Artificial Sequence Synthetic peptide
TALVPR

SEQ ID NO: 63 - protein, 6 aa, Artificial Sequence
  Description of Artificial Sequence Synthetic peptide
TVLPAR

SEQ ID NO: 64 - protein, 6 aa, Artificial Sequence
  Description of Artificial Sequence Synthetic peptide
TLVPAR

SEQ ID NO: 65 - protein, 7 aa, Artificial Sequence
  Description of Artificial Sequence Synthetic peptide
TAVSLKE

SEQ ID NO: 66 - protein, 7 aa, Artificial Sequence
  Description of Artificial Sequence Synthetic peptide
TALVSKE

SEQ ID NO: 67 - protein, 7 aa, Artificial Sequence
  Description of Artificial Sequence Synthetic peptide
TVLSAKE

SEQ ID NO: 68 - protein, 7 aa, Artificial Sequence
  Description of Artificial Sequence Synthetic peptide
TLVSAKE

SEQ ID NO: 69 - protein, 7 aa, Artificial Sequence
  Description of Artificial Sequence Synthetic peptide
TMVPLKE

SEQ ID NO: 70 - protein, 7 aa, Artificial Sequence
  Description of Artificial Sequence Synthetic peptide
TMLVPKE

SEQ ID NO: 71 - protein, 7 aa, Artificial Sequence
  Description of Artificial Sequence Synthetic peptide
TVLPMKE

SEQ ID NO: 72 - protein, 7 aa, Artificial Sequence
  Description of Artificial Sequence Synthetic peptide
TLVPMKE

SEQ ID NO: 73 - protein, 7 aa, Artificial Sequence
  Description of Artificial Sequence Synthetic peptide
TTVPLKD

SEQ ID NO: 74 - protein, 7 aa, Artificial Sequence
  Description of Artificial Sequence Synthetic peptide
TTLVPKD

SEQ ID NO: 75 - protein, 7 aa, Artificial Sequence
  Description of Artificial Sequence Synthetic peptide
TVLPTKD

SEQ ID NO: 76 - protein, 7 aa, Artificial Sequence
  Description of Artificial Sequence Synthetic peptide
TLVPTKD

SEQ ID NO: 77 - protein, 7 aa, Artificial Sequence
  Description of Artificial Sequence Synthetic peptide
TAVPLRD

SEQ ID NO: 78 - protein, 7 aa, Artificial Sequence
  Description of Artificial Sequence Synthetic peptide
TALVPRD

SEQ ID NO: 79 - protein, 7 aa, Artificial Sequence
  Description of Artificial Sequence Synthetic peptide
TVLPARD

SEQ ID NO: 80 - protein, 7 aa, Artificial Sequence
  Description of Artificial Sequence Synthetic peptide
TLVPARD

SEQ ID NO: 81 - protein, 7 aa, Artificial Sequence
  Description of Artificial Sequence Synthetic peptide
TAVSLFK

SEQ ID NO: 82 - protein, 7 aa, Artificial Sequence
  Description of Artificial Sequence Synthetic peptide
TALVSFK

SEQ ID NO: 83 - protein, 7 aa, Artificial Sequence
  Description of Artificial Sequence Synthetic peptide
TVLSAFK

SEQ ID NO: 84 - protein, 7 aa, Artificial Sequence
  Description of Artificial Sequence Synthetic peptide
TLVSAFK

SEQ ID NO: 85 - protein, 7 aa, Artificial Sequence
  Description of Artificial Sequence Synthetic peptide
TMVPLFK

SEQ ID NO: 86 - protein, 7 aa, Artificial Sequence
  Description of Artificial Sequence Synthetic peptide
TMLVPFK

SEQ ID NO: 87 - protein, 7 aa, Artificial Sequence
  Description of Artificial Sequence Synthetic peptide
TVLPMFK

SEQ ID NO: 88 - protein, 7 aa, Artificial Sequence
  Description of Artificial Sequence Synthetic peptide
TLVPMFK

SEQ ID NO: 89 - protein, 7 aa, Artificial Sequence
  Description of Artificial Sequence Synthetic peptide
TTVPLFK

SEQ ID NO: 90 - protein, 7 aa, Artificial Sequence
  Description of Artificial Sequence Synthetic peptide
TTLVPFK

SEQ ID NO: 91 - protein, 7 aa, Artificial Sequence
  Description of Artificial Sequence Synthetic peptide
TVLPTFK

SEQ ID NO: 92 - protein, 7 aa, Artificial Sequence
  Description of Artificial Sequence Synthetic peptide
TLVPTFK

SEQ ID NO: 93 - protein, 7 aa, Artificial Sequence
  Description of Artificial Sequence Synthetic peptide
TAVPLFR

SEQ ID NO: 94 - protein, 7 aa, Artificial Sequence
  Description of Artificial Sequence Synthetic peptide
TALVPFR

SEQ ID NO: 95 - protein, 7 aa, Artificial Sequence
  Description of Artificial Sequence Synthetic peptide
TVLPAFR

SEQ ID NO: 96 - protein, 7 aa, Artificial Sequence
  Description of Artificial Sequence Synthetic peptide
TLVPAFR

SEQ ID NO: 97 - protein, 5 aa, Artificial Sequence
  Description of Artificial Sequence Synthetic peptide
  MOD_RES (1)..(1): V, A, L, I, G, P, S, T, or M
  MOD_RES (2)..(2): V, A, L, I, G, P, S, T, or M
  MOD_RES (3)..(3): V, A, L, I, G, P, S, T, or M
  MOD_RES (4)..(4): V, A, L, I, G, P, S, T, or M
  MOD_RES (5)..(5): K, R, H, D, or E
  See specification as filed for detailed description of substitutions and preferred embodiments
XXXXX

SEQ ID NO: 98 - protein, 6 aa, Artificial Sequence
  Description of Artificial Sequence Synthetic peptide
  MOD_RES (2)..(2): V, A, L, I, G, P, S, T, or M
  MOD_RES (3)..(3): V, A, L, I, G, P, S, T, or M
  MOD_RES (4)..(4): V, A, L, I, G, P, S, T, or M
  MOD_RES (5)..(5): V, A, L, I, G, P, S, T, or M
  MOD_RES (6)..(6): K, R, H, D, or E
  See specification as filed for detailed description of substitutions and preferred embodiments
TXXXXX

SEQ ID NO: 99 - protein, 6 aa, Artificial Sequence
  Description of Artificial Sequence Synthetic peptide
  MOD_RES (1)..(1): V, A, L, I, G, P, S, T, or M
  MOD_RES (2)..(2): V, A, L, I, G, P, S, T, or M
  MOD_RES (3)..(3): V, A, L, I, G, P, S, T, or M
  MOD_RES (4)..(4): V, A, L, I, G, P, S, T, or M
  MOD_RES (5)..(5): K, R, H, D, or E
  MOD_RES (6)..(6): E or D
  See specification as filed for detailed description of substitutions and preferred embodiments
XXXXXX

SEQ ID NO: 100 - protein, 7 aa, Artificial Sequence
  Description of Artificial Sequence Synthetic peptide
  MOD_RES (1)..(1): F, L, W, or Y
  MOD_RES (3)..(3): V, A, L, I, G, P, S, T, or M
  MOD_RES (4)..(4): V, A, L, I, G, P, S, T, or M
  MOD_RES (5)..(5): V, A, L, I, G, P, S, T, or M
  MOD_RES (6)..(6): V, A, L, I, G, P, S, T, or M
  MOD_RES (7)..(7): K, R, H, D, or E
  See specification as filed for detailed description of substitutions and preferred embodiments
XTXXXXX

SEQ ID NO: 101 - protein, 7 aa, Artificial Sequence
  Description of Artificial Sequence Synthetic peptide
  MOD_RES (2)..(2): V, A, L, I, G, P, S, T, or M
  MOD_RES (3)..(3): V, A, L, I, G, P, S, T, or M
  MOD_RES (4)..(4): V, A, L, I, G, P, S, T, or M
  MOD_RES (5)..(5): V, A, L, I, G, P, S, T, or M
  MOD_RES (6)..(6): K, R, H, D, or E
  MOD_RES (7)..(7): E or D
  See specification as filed for detailed description of substitutions and preferred embodiments
TXXXXXX

SEQ ID NO: 102 - protein, 7 aa, Artificial Sequence
  Description of Artificial Sequence Synthetic peptide
  MOD_RES (1)..(1): V, A, L, I, G, P, S, T, or M
  MOD_RES (2)..(2): V, A, L, I, G, P, S, T, or M
  MOD_RES (3)..(3): V, A, L, I, G, P, S, T, or M
  MOD_RES (4)..(4): V, A, L, I, G, P, S, T, or M
  MOD_RES (5)..(5): K, R, H, D, or E
  MOD_RES (6)..(6): E or D
  MOD_RES (7)..(7): A or I
  See specification as filed for detailed description of substitutions and preferred embodiments
XXXXXXX

SEQ ID NO: 103 - protein, 4 aa, Artificial Sequence
  Description of Artificial Sequence Synthetic peptide
  MOD_RES (2)..(2): S or P
  MOD_RES (3)..(3): A, M, or T
  See specification as filed for detailed description of substitutions and preferred embodiments
VXXL

SEQ ID NO: 104 - protein, 5 aa, Artificial Sequence
  Description of Artificial Sequence Synthetic peptide
  MOD_RES (3)..(3): S or P
  MOD_RES (4)..(4): A, M, or T
  See specification as filed for detailed description of substitutions and preferred embodiments
TVXXL

SEQ ID NO: 105 - protein, 6 aa, Artificial Sequence
  Description of Artificial Sequence Synthetic peptide
  MOD_RES (3)..(3): S or P
  MOD_RES (4)..(4): A, M, or T
  See specification as filed for detailed description of substitutions and preferred embodiments
TVXXLK

SEQ ID NO: 106 - protein, 7 aa, Artificial Sequence
  Description of Artificial Sequence Synthetic peptide
  MOD_RES (3)..(3): S or P
  MOD_RES (4)..(4): A, M, or T
  See specification as filed for detailed description of substitutions and preferred embodiments
TVXXLFK

SEQ ID NO: 107 - protein, 6 aa, Artificial Sequence
  Description of Artificial Sequence Synthetic peptide
KLASVT

SEQ ID NO: 108 - protein, 7 aa, Artificial Sequence
  Description of Artificial Sequence Synthetic peptide
KFLASVT

SEQ ID NO: 109 - RNA, 22 nt, Homo sapiens
acauacuucuuuauaugcccau

SEQ ID NO: 110 - DNA, 22 bp, Mus musculus
atcaaggtccgctgtgaacacg

SEQ ID NO: 111 - DNA, 22 bp, Mus musculus
caaacaccattgtcacactcca

SEQ ID NO: 112 - DNA, 22 bp, Mus musculus
ccaaacactgctgggtaagacg

SEQ ID NO: 113 - RNA, 22 nt, Mus musculus
cguguucacagcggaccuugau

SEQ ID NO: 114 - RNA, 22 nt, Mus musculus
uggagugugacaaugguguuug

SEQ ID NO: 115 - RNA, 22 nt, Mus musculus
cgucuuacccagcaguguuugg